(19) United States
(12) Patent Application Publication
(10) Pub. No.: US 2025/0059567 A1
(43) Pub. Date: Feb. 20, 2025

(54) COMPOSITIONS AND METHODS FOR THE MANAGEMENT AND TREATMENT OF PHENYLKETONURIA

(71) Applicants: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(72) Inventors: Kiran Musunuru, Philadelphia, PA (US); Xiao Wang, Merion Station, PA (US); Dominique Lynnette Brooks, Philadelphia, PA (US); Rebecca Ahrens-Nicklas, Haverford, PA (US)

(73) Assignees: THE TRUSTEES OF THE UNIVERSITY OF PENNSYLVANIA, Philadelphia, PA (US); THE CHILDREN'S HOSPITAL OF PHILADELPHIA, Philadelphia, PA (US)

(21) Appl. No.: 18/390,476

(22) Filed: Dec. 20, 2023

Related U.S. Application Data
(60) Provisional application No. 63/520,273, filed on Aug. 17, 2023.

(51) Int. Cl. C12N 15/90; C12N 9/22; C12N 9/78; C12N 15/11

(52) U.S. Cl. CPC C12N 15/90; C12N 9/22; C12N 9/78; C12N 15/11; C12Y 305/04004; C12N 2310/20

(57) ABSTRACT

Compositions and methods for effecting base editing to correct mutations in the phenylalanine hydroxylase gene, thereby curing phenylketonuria, are disclosed.

### CROSS-REFERENCE TO RELATED APPLICATION

This application claims priority of U.S. Provisional application No. 63/520,273 filed Aug. 17, 2023, the entire contents being incorporated herein by reference as though set forth in full.

### GRANT SUPPORT STATEMENT

This invention was made with government support under NS132301, HL148769, and HL145203 awarded by the National Institutes of Health. The government has certain rights in the invention.

### FIELD OF THE INVENTION

This invention relates to the fields of genetic engineering and correction of genetic errors using base editing therapy. More specifically, the invention provides compositions and methods for correcting gene mutations which cause phenylketonuria.

### INCORPORATION BY REFERENCE OF MATERIAL SUBMITTED IN ELECTRONIC FORM

The Contents of the electronic sequence listing (UPNK-117-US.xml; Size: 25,401 bytes; and Date of Creation: Aug. 17, 2023) is herein incorporated by reference in its entirety.

### BACKGROUND OF THE INVENTION

Several publications and patent documents are cited throughout the specification in order to describe the state of the art to which this invention pertains. Each of these citations is incorporated herein by reference as though set forth in full.

Phenylketonuria (PKU) is an autosomal recessive inborn error of metabolism caused by a deficiency in the hepatic enzyme phenylalanine hydroxylase (PAH). If left untreated, the main clinical feature is intellectual disability. Treatment, which includes a low phenylalanine diet supplemented with amino acid formulas, commences soon after diagnosis within the first weeks of life. Although dietary treatment has been successful in preventing intellectual disability in early treated PKU patients, there are major issues with dietary compliance due to the palatability of the diet. Other potential issues associated with dietary therapy include nutritional deficiencies particularly in vitamins D and B12. Suboptimal outcomes in cognitive and executive functioning have been reported in patients who adhere poorly to dietary therapy.

Other approaches include administration of oral medication, e.g., sapropterin, a cofactor of PAH, and an injectable enzyme substitution therapy (pegvaliase). Many PKU patients have limited responses to, or limited access to the medical therapies and, as a result, have impaired cognitive development and develop a range of neuropsychiatric problems. Durable and, ideally, curative therapies are needed to address the unmet medical needs of PKU patients. Although the liver is spared from toxicity, the PAH gene is largely expressed in hepatocytes, and correction of the primary genetic defect solely within the liver would be curative in PKU patients.

It is an object of the invention to provide an effective and lasting treatment of PKU which reduces or eliminates PKU symptoms.

### SUMMARY OF THE INVENTION

The present invention provides compositions and methods for effecting a durable cure of a subset of patients with phenylketonuria via the direct correction of causative mutations for this disease, particularly the c.1222C>T mutation, also known as p.Arg408Trp mutation and R408W mutation, which is the most common mutation associated with PKU worldwide.

In accordance with one aspect of the invention a method for editing a phenylalanine hydroxylase (PAH) encoding polynucleotide comprising a c.1222C>T (p.Arg408Trp) mutation is provided. An exemplary method comprises contacting the PAH polynucleotide with a base editor in complex with at least one guide polynucleotide, wherein the base editor comprises a polynucleotide programmable DNA binding domain and an adenosine deaminase domain, and wherein one or more of said guide polynucleotides target said base editor to effect an A·T to G·C alteration of the mutation thereby restoring the wild-type sequence and correcting the disease phenotype. In certain embodiments, the contacting is done in a cell, a eukaryotic cell, a mammalian cell, or human cell. Contacting may be performed in vitro or in vivo. In certain embodiments, the patient has a second mutation in a different allele in a second PAH encoding polynucleotide selected from c.842C>T (p.Pro281Leu), c.1066-11G>A, c.782G>A (p.Arg261Gln), c.728G>A (p.Arg243Gln), c.1315+1G>A, and c.473G>A (p.Arg158Gln). The polynucleotide programmable DNA binding domain can be a Streptococcus pyogenes Cas9 (SpCas9) or Staphylococcus aureus Cas9 (SaCas9) or a variant thereof. In certain aspects, the polynucleotide programmable DNA binding domain comprises a modified SpCas9 having an altered protospacer-adjacent motif (PAM) specificity, including without limitation, a modified SpCas9 having specificity for the nucleic acid sequence 5′-NGC-3′, 5′-NCA-3′, 5′-NAA-3′, 5′-NAG-3′, 5′-NGT-3′, or 5′-NGN-3′. The polynucleotide programmable DNA binding domain may be nuclease inactive or nickase variant. In the base editing methods disclosed the adenosine deaminase domain is capable of deaminating adenosine in deoxyribonucleic acid (DNA). The adenosine deaminase can be a TadA deaminase or a variant thereof. In another embodiment, the base editor is in complex with a single guide RNA (sgRNA) comprising a nucleic acid sequence complementary to a nucleic acid sequence comprising the mutation associated with PKU.

Also provided is a cell comprising a base editor, or a polynucleotide encoding said base editor, wherein said base editor comprises a polynucleotide programmable DNA binding domain and an adenosine deaminase domain; and one or more guide polynucleotides that target the base editor to effect an A·T to G·C alteration of a c.1222C>T (p.Arg408Trp) mutation associated with PKU. In a preferred embodiment, the cell is a hepatocyte obtained from a subject having PKU and expresses PAH polypeptide.

Another embodiment of the invention comprises an adenosine base editor/guide polynucleotide set which corrects a mutation causing PKU. An exemplary set includes (i) a modified SpCas9 or SaCas9; (ii) an adenosine deaminase or functional fragment thereof; and (iii) a guide polynucleotide that targets the base editor to effect an A·T to G·C alteration of alteration of a c.1222C>T (p.Arg408Trp) mutation associated with PKU. In preferred embodiments, the guide polynucleotide has a sequence of SEQ ID NO: 1, SEQ ID NO: 3, SEQ ID NO: 5, SEQ ID NO: 7, SEQ ID NO: 9, or SEQ ID NO: 11. In other embodiments, the guide polynucleotide comprises a nucleic acid sequence complementary to a PAH encoding nucleic acid sequence of SEQ ID NO: 13, SEQ ID NO: 14, SEQ ID NO: 15, SEQ ID NO: 16, SEQ ID NO: 17, or SEQ ID NO: 18. The guide polynucleotide can be RNA or DNA or a combination thereof. In particularly preferred embodiments, the guide polynucleotide has a sequence of SEQ ID NO: 7. In another preferred embodiment. The base editor has a sequence of SEQ ID NO: 20.

Another aspect of the invention includes a method of treating PKU in a subject comprising administering to said subject an effective amount of the adenosine base editor/guide polynucleotide sets described above. Subjects to be treated include mammals and humans. The base editor, or polynucleotide encoding said base editor, and said one or more guide polynucleotides can be delivered to a cell of the subject, particularly a liver cell.

In one delivery method, the base editor/guide polynucleotide set can be encapsulated in a lipid nanoparticle formulation and delivered to the liver of said subject. In certain aspects, the formulation comprises ionizable cationic lipid, 1,2-distearoyl-sn-glycero-3-phosphocholine, cholesterol, and a PEG-lipid. In an alternative delivery method, the base editor/guide polynucleotide set is delivered to hepatocytes in a single or dual AAV vector system as described herein. In yet another approach, the base editor/guide polynucleotide set can be delivered to hepatocytes in vivo or in vitro in virus-like particles.

In another aspect of the invention, a transgenic mouse comprising a humanized PAH gene comprising a c.1222C>T (p.Arg408Trp) mutation. In certain embodiments, the mouse further comprises at least one mutation selected from c.842C>T (p.Pro281Leu), c.1066-11G>A, c.782G>A (p.Arg261Gln), c.728G>A (p.Arg243Gln), c.1315+1G>A, and c.473G>A (p.Arg158Gln).

Still other aspects and advantages of these compositions and methods are readily apparent and described further in the following detailed description of the invention.

### BRIEF DESCRIPTION OF THE DRAWINGS

FIGS. 1A-1B. (FIG. 1A) Adenine base editors (ABEs) are composed of a dead (d) or nickase (n) Cas9 (d/nCas9) fused to one or two TadA, either a TadA evolved to edit adenine in DNA (TadA*) or both a TadA* and a wild-type TadA. ABEs convert A:T into G:C base pairs in the editing window (for example, nucleotide 4 to 7 in the protospacer, in purple). Cas9 is guided by the sgRNA to the protospacer [which is followed by the PAM (protospacer adjacent motif)] and unwinds the DNA, and the deaminase converts the target base. (FIG. 1B) Cytosine base editors (CBEs), composed of a nickase Cas9 (nCas9) fused to a deaminase and one (in BE3s) or two (in BE4s) UGI (uracil glycosylase inhibitor), convert C·G into T·A base pairs in the editing window (nucleotide 4 to 8 in the protospacer, in green). Undesired events (bystander edits, in blue, and unwanted base conversion, in yellow) of CBEs and ABEs are shown in (FIGS. 1A, 1B), respectively. The addition of the second UGI in CBEs (in BE4) and the removal of TadA in ABEs (ABE8) are highlighted with a gray dotted line. The gradient color of the editing window in the upper panels of (FIGS. 1A, 1B) represents the enlarged editing window observed with novel BEs.

FIG. 2. Lifetime recorded measurements of blood phenylalanine levels in PKU patients with PAH R408W variants.

FIG. 3. Intervals between measurements of blood phenylalanine levels in PKU patients with PAH R408W variants.

FIG. 4A-4C. (FIG. 4A) Schematic of the genomic site of the PAH c.1222C>T (R408W) variant, adapted from the UCSC Genome Browser (GRCh38/hg38)(SEQ ID NO: 23). The vertical blue bar indicates the G altered to A (in red) by the variant on the antisense strand. The target protospacers for six gRNAs designated PAH1 (SEQ ID NO: 2), PAH2 (SEQ ID NO: 4), PAH3 (SEQ ID NO: 6), PAH4 (SEQ ID NO: 8), PAH5 (SEQ ID NO: 10), and PAH6 (SEQ ID NO: 12) are indicated with thick green horizontal bars, with the thin portions of the bars indicating PAMs. The black arrows indicate sites of potential bystander adenine editing. (FIG. 4B) Sanger sequencing chromatogram of the genomic site of the PAH R408W variant in wild-type HuH-7 cells. The blue arrow indicates the position of the variant. (FIG. 4C) Chromatogram of the site in R408W homozygous HuH-7 cell line. The red arrow indicates the position of the variant.

FIG. 5A-5B. Adenine base editing to correct PAH c.1222C>T variant in HuH-7 human hepatoma cells. (FIG. 5A) Corrective A-to-G editing following transient transfection of R408W homozygous HuH-7 cells with plasmids encoding varied ABE/gRNA sets. (FIG. 5B) Sanger sequencing chromatogram of the genomic site of the variant in R408W homozygous HuH-7 cells treated with SpRY-ABE8e/PAH4 set. The blue arrow indicates the position of the variant.

FIG. 6. A-to-G editing observed in dose-response study with R408W homozygous HuH-7 cells treated with LNPs formulated with SpRY-ABE8.8 mRNA and PAH4 gRNA. The editing percentage includes all outcomes in which corrective editing of the R408W variant was achieved, irrespective of bystander editing (n=3 biological replicates; mean±standard deviation for each dose).

FIG. 7. Scheme to generate exon-humanized PKU mice with the PAH c.1222C>T (R408W) variant.

FIGS. 8A-8B. Generation of minimally humanized PKU mice with the PAH c.1222C>T (R408W) variant. (FIG. 7A) Sanger sequencing chromatograms showing the generation of a humanized mouse model via Cas9-mediated homology-directed repair in mouse zygotes. At the top is sequence from a wild-type C57BL/6J mouse. At the bottom is sequence from a mouse homozygous for the humanized R408W allele. The red arrow indicates the site of the R408W variant, and the black arrows indicate the sites of synonymous changes that humanize the local region of the mouse Pah gene. (FIG. 7B) Age-matched littermates that are homozygous or heterozygous for the humanized R408W allele. On the left is a homozygous mice with PKU as evidenced by hypopigmentation of the fur, and on the right is a control heterozygous mice with normal fur color.

FIGS. 9A-9B. Adenine base editing for correction of the PAH R408W variant in humanized PKU mice in vivo. (FIG. 9A) Short-term changes in the blood phenylalanine level in homozygous PKU mice following treatment with SpRY-ABE8.8/PAH4 LNPs, comparing levels at various time points up to 7 days following treatment to levels in untreated control non-PKU colony mates (n=1 sample per animal at each time point). (FIG. 9B) A-to-G editing of the PAH R408W variant in the whole liver (n=8 samples per animal).

### DETAILED DESCRIPTION OF THE INVENTION

In vivo gene editing is an emerging therapeutic approach to making DNA modifications in the body of a patient, such as in the liver. Gene-editing methods include CRISPR-Cas9 and -Cas12 nucleases, CRISPR cytosine base editors, CRISPR adenine base editors, and CRISPR prime editors. CRISPR base editors are an attractive gene-editing modality because they function efficiently for introducing precise targeted alterations without the need for double-strand breaks, in contrast to CRISPR-Cas9 and other gene-editing nucleases (e.g., Cas12). Adenine base editors (ABEs) can induce targeted A→G edits in DNA (T→C on the opposing strand). Each ABE uses its core Cas9 nickase protein with a guide RNA (gRNA) to engage a double-strand protospacer DNA sequence, flanked by a protospacer-adjacent motif (PAM) sequence on its 3′ end. Because ABEs do not make double-strand breaks, they have a minimal risk of inducing large deletions, chromosomal abnormalities, and chromothripsis (shattering); instead, each ABE uses an evolved deoxyadenosine deaminase domain—typically fused to the N-terminal end of the Cas9 nickase—to chemically modify an adenosine nucleoside on one DNA strand, which (in combination with nicking of the other strand) enables highly precise and efficient A→G transition mutations at the targeted site.

The activity window of each ABE typically ranges across several positions within the protospacer DNA sequence (e.g., the ABE8.8 window ranges from position 3 to position 9, with peak editing observed at position 6 of the protospacer), with different ABEs having different windows. ABEs have the potential to edit any adenine within the window, which could include a desired target adenine but also undesired additional adenines (bystander edits). Published ABEs with Streptococcus pyogenes Cas9 nickase include so-called eighth-generation ABEs (harboring optimized deaminase domains resulting from eight rounds of molecular evolution)—the most commonly used to date are ABE8.8, ABE8.20, and ABE8e—and circularly permuted or inlaid ABEs, in which the deaminase domain is embedded within a loop of the Cas9 nickase protein, rather than fused to the N-terminal end, which has the effect of shifting the editing window further towards the 3′ end of the protospacer sequence. Similar ABEs with Cas9 nickase from other bacterial species (e.g., Staphylococcus aureus) have been reported. As a general rule, ABEs display highly variable levels of activity across different genomic loci in different cell types, and empirical testing is mandatory to determine whether a given ABE with a given gRNA will edit efficiently at a given target site in a given cell type.

The present invention provides compositions and methods for adenine base editing to permanently correct the most common pathogenic variant, the PAH c.1222C>T (R408W) variant, in human hepatocytes. The PAH c.1222C>T variant has its highest prevalence in populations in Eastern Europe but is widespread across the globe and affects a large proportion of PKU patients in the United States. Patients homozygous for this variant do not typically respond to sapropterin, limiting their treatment options and making a curative in vivo base editing therapy particularly compelling.

### Definitions

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

A “monogenic disease” or a “monogenic disorder” is a condition determined by the interaction of a single pair of genes. This is in contrast to a polygenic condition wherein several genes are involved. In humans, monogenic diseases occur less frequently than the polygenic disease. It is also less complicated than the latter and may follow a pattern based on Mendelian inheritance. Monogenic disorders can adversely impact a number of biological systems.

Phenylketonuria (PKU) is a classic “monogenic” autosomal recessive disease in which mutation at the human phenylalanine hydroxylase (PAH) locus impairs the function of the enzyme phenylalanine hydroxylase (enzymic phenotype), thereby causing the attendant hyperphenylalaninemia (metabolic phenotype) and the resultant intellectual disability (cognitive phenotype). Other symptoms include seizures, tremors, hyperactivity, stunted growth, or shaking and trembling, skin conditions including eczema, as well as musty odor of the urine, breath, or skin. 0.45 million individuals have PKU, with global prevalence 1:23,930 live births (range 1:4,500 [Italy]-1:125,000 [Japan]). More than 1280 variants in the phenylalanine hydroxylase PAH gene are responsible for a broad spectrum of phenylketonuria (PKU) phenotypes. While genotype-phenotype correlation is ˜88%, additional factors play a role. These include tetrahydrobiopterin (BH₄), the PAH co-chaperone DNAJC12, phosphorylation of the PAH residues, and epigenetic factors. There is presently no cure for PKU, with the exception of liver transplantation. Here the direct correction of the causative mutation PAH c.1222C>T via base editing, also known as p.Arg408Trp and R408W, in liver cells is described. This is the most common PKU associated gene mutation worldwide. Subjects harboring this mutation do not typically respond well to BH₄ supplementation therapy.

The term “deaminase” or “deaminase domain” refers to a protein or enzyme that catalyzes a deamination reaction. In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium.

In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75%, at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine.

It should be appreciated, however, that additional adenosine deaminases useful in the present application would be apparent to the skilled artisan and are within the scope of this disclosure.

The term “base editor (BE)” or “nucleobase editor (NBE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating an adenine (A) in DNA. In some embodiments, the base editor is a fusion protein comprising a nucleic acid programmable DNA binding protein (napDNAbp) fused to an adenosine deaminase. In some embodiments, the base editor is a Cas9 protein fused to an adenosine deaminase. In some embodiments, the base editor is a Cas9 nickase (nCas9) fused to an adenosine deaminase. In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain.

“Prime editing” directly introduces new genetic information into a targeted DNA site. Typically editing is effected by a fusion protein, consisting of a catalytically impaired Cas9 endonuclease fused to an engineered reverse transcriptase enzyme, and a prime editing guide RNA (pegRNA), capable of identifying the target site and providing the new genetic information to replace the target DNA nucleotides. Using this technique targeted insertions, deletions, and base-to-base conversions without the need for double strand breaks (DSBs) or donor DNA templates can be introduced into the targeted nucleic acid molecule.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

As used herein the term “wild-type” is a term of the art understood by skilled persons and means the typical form of an organism, strain, gene, or characteristic as it occurs in nature as distinguished from mutant or variant forms. As used herein the term “variant” should be taken to mean the exhibition of qualities that have a pattern that deviates from the wild-type or comprises non naturally occurring components.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4.sup.th ed., Cold Spring Harbor Laboratory Press, Cold Spring Harbor, N.Y. (2012)).

The term “uracil glycosylase inhibitor” or “UGI,” as used herein, refers to a protein that is capable of inhibiting a uracil-DNA glycosylase base-excision repair enzyme. In some embodiments, the UGI proteins provided herein include fragments of UGI and proteins homologous to a UGI or a UGI fragment. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence encoding UGI.

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for their disclosure of exemplary nuclear localization sequences.

The term “nucleic acid programmable DNA binding protein” or “napDNAbp” refers to a protein that associates with a nucleic acid (e.g., DNA or RNA), such as a guide nucleic acid, that guides the napDNAbp to a specific nucleic acid sequence. For example, a Cas9 protein can associate with a guide RNA that guides the Cas9 protein to a specific DNA sequence that has complementarity to the spacer sequence in the guide RNA. In some embodiments, the napDNAbp is a class 2 microbial CRISPR-Cas effector. In some embodiments, the napDNAbp is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, C2c1, C2c2, C2C3, and Argonaute. It should be appreciated, however, that nucleic acid programmable DNA binding proteins also include nucleic acid programmable proteins that bind RNA. For example, the napDNAbp may be associated with a nucleic acid that guides the napDNAbp to an RNA. Other nucleic acid programmable DNA binding proteins are also within the scope of this disclosure, though they may not be specifically listed in this disclosure.

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). CRISPR (clustered regularly interspaced short palindromic repeat) is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc), and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif present in the target DNA sequence (the PAM or protospacer-adjacent motif) but not in the CRISPR repeat sequences to help distinguish non-self versus self, since the presence of the PAM is required for the binding activity and catalytic activity of Cas9. Cas9 nuclease sequences and structures are well known to those of skill in the art (see, e.g., Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E. “Complete genome sequence of an M1 strain of Streptococcus pyogenes.” Proc. Natl. Acad. Sci. U.S.A. 98:4658-4663 (2001); Deltcheva E., Chylinski K., Sharma C. M., Gonzales K., Chao Y., Pirzada Z. A., Eckert M. R., Vogel J., Charpentier E. “CRISPR RNA maturation by trans-encoded small RNA and host factor RNase III.” Nature 471:602-607(2011); and Jinek M., Chylinski K., Fonfara I., Hauer M., Doudna J. A., Charpentier E. “A programmable dual-RNA-guided DNA endonuclease in adaptive bacterial immunity.” Science 337:816-821 (2012); the entire contents of each of which are incorporated herein by reference). Cas9 orthologs have been described in various species, including, but not limited to, Streptococcus pyogenes (S. pyogenes Cas9 or SpCas9) and Staphylococcus aureus (S. aureus Cas9 or SaCas9). Additional suitable Cas9 nucleases and sequences will be apparent to those of skill in the art based on this disclosure, and such Cas9 nucleases and sequences include Cas9 sequences from the organisms and loci disclosed in Chylinski K., Rhun A. L., and Charpentier E., “The tracrRNA and Cas9 families of type II CRISPR-Cas immunity systems.” RNA Biol. 10:726-737 (2013); the entire contents of which are incorporated herein by reference. In some embodiments, a Cas9 nuclease has an inactive (e.g., an inactivated) DNA cleavage domain.

In some embodiments, Cas9 is adapted from Streptococcus pyogenes (S. pyogenes; GenBank Ref: AP014596.1). In certain embodiments the Cas9 requires a PAM comprising the sequence NGG in the target DNA sequence for it to have binding activity and catalytic activity. In some embodiments, Cas9 is further engineered to require a PAM differing from the sequence NGG. In some embodiments, Cas9 is further engineered to not require a specific PAM (see, e.g., Walton R. T., Christie K. A., Whittaker M. N., Kleinstiver B. P. “Unconstrained genome targeting with near-PAMless engineered CRISPR-Cas9 variants.” Science 368:290-296 (2020); the entire contents of which are incorporated herein by reference). In certain embodiments, the Cas9 is adapted from S. pyogenes and requires a PAM comprising the sequence NGG in the target DNA sequence, is further engineered to require a PAM differing from the sequence NGG, or is further engineered to not require a specific PAM.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (see, e.g., Jinek et al., Science. 337:816-821 (2012); Qi L. S., Larson M. H., Gilbert L. A., Doudna J. A., Weissman J. S., Arkin A. P., Lim W. A. “Repurposing CRISPR as an RNA-guided platform for sequence-specific control of gene expression.” Cell 152:1173-1183 (2013); the entire contents of each of which are incorporated herein by reference).

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1), or Neisseria meningitidis (NCBI Ref: YP_002342100.1) or to a Cas9 from any other organism.

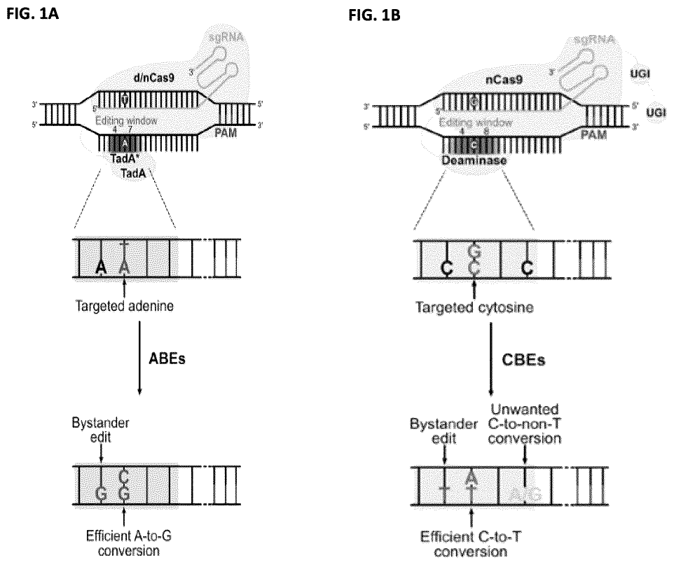

In some embodiments, dCas9 corresponds to, or comprises in part or in whole, a Cas9 amino acid sequence having one or more mutations that inactivate the Cas9 nuclease activity. Such Cas9 variants are able to generate a single-strand DNA break (nick) at a specific location based on the gRNA-defined target sequence, leading to repair of the non-edited strand, ultimately resulting in a T to C change on the non-edited strand. A schematic representation of this process is shown in FIG. 1A. Briefly, and without wishing to be bound by any particular theory, the A of a A-T base pair can be deaminated to an inosine (I) by an adenosine deaminase, e.g., an engineered adenosine deaminase that deaminates an adenosine in DNA. Nicking the non-edited strand, having the T, facilitates removal of the T via mismatch repair mechanisms. A catalytically inactive inosine-specific nuclease (dISN) may inhibit inosine-specific nucleases (e.g., sterically) thereby preventing removal of the inosine (I).

In some embodiments, the nucleic acid programmable DNA binding protein (napDNAbp) of any of the fusion proteins provided herein may be a CasX or CasY protein. In some embodiments, the napDNAbp is a CasX protein. In some embodiments, the napDNAbp is a CasY protein.

The term “effective amount,” as used herein, refers to an amount of a biologically active agent that is sufficient to elicit a desired biological response. For example, in some embodiments, an effective amount of a nucleobase editor may refer to the amount of the nucleobase editor that is sufficient to induce mutation of a target site specifically bound and mutated by the nucleobase editor. In some embodiments, an effective amount of a fusion protein provided herein, e.g., of a fusion protein comprising a nucleic acid programmable DNA binding protein and a deaminase domain (e.g., an adenosine deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nucleobase editor, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides.

Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4.sup.th ed., Cold Spring Harbor Laboratory Press, Cold Spring Harbor, N.Y. (2012)), the entire contents of which are incorporated herein by reference.

The term “RNA-programmable nuclease” and “RNA-guided nuclease” are used interchangeably herein and refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target), which may be referred to as a spacer or a spacer sequence; and (2) a domain that binds a Cas9 protein. In some embodiments, domain (2) corresponds to a sequence known as a tracrRNA and comprises a stem-loop structure. For example, in some embodiments, domain (2) is identical or homologous to a tracrRNA as provided in Jinek et al., Science 337:816-821 (2012), the entire contents of which is incorporated herein by reference. Other examples of gRNAs (e.g., those including domain 2) can be found in U.S. Provisional Patent Application Ser. No. 61/874,682, filed Sep. 6, 2013, entitled “Switchable Cas9 Nucleases And Uses Thereof,” and U.S. Provisional Patent Application Ser. No. 61/874,746, filed Sep. 6, 2013, entitled “Delivery System For Functional Nucleases,” the entire contents of each are hereby incorporated by reference in their entirety. Exemplary gRNAs and target sites are identified in Table 1.

TABLE 1

| | | SEQ |
|---|---|---|
| Sequence | | ID |
| Type | Sequence | NO: |
| gRNA | aagggccaagguauuguggcguuuuagagcuaga | 1 |
| | aauagcaaguuaaaauaaggcuaguccguuauca | |
| | acuugaaaaaguggcaccgagucggugcuuuu | |
| gRNA | agggccaagguauuguggcaguuuuagagcuaga | 3 |
| | aauagcaaguuaaaauaaggcuaguccguuauca | |
| | acuugaaaaaguggcaccgagucggugcuuuu | |
| gRNA | gggccaagguauuguggcagguuuuagagcuaga | 5 |
| | aauagcaaguuaaaauaaggcuaguccguuauca | |
| | acuugaaaaaguggcaccgagucggugcuuuu | |
| gRNA | ggccaagguauuguggcagcguuuuagagcuaga | 7 |
| | aauagcaaguuaaaauaaggcuaguccguuauca | |
| | acuugaaaaaguggcaccgagucggugcuuuu | |
| gRNA | gccaagguauuguggcagcaguuuuagagcuaga | 9 |
| | aauagcaaguuaaaauaaggcuaguccguuauca | |
| | acuugaaaaaguggcaccgagucggugcuuuu | |
| gRNA | ccaagguauuguggcagcaaguuuuagagcuaga | 11 |
| | aauagcaaguuaaaauaaggcuaguccguuauca | |
| | acuugaaaaaguggcaccgagucggugcuuuu | |
| PAH1 | aagggccaaggtattgtggc | 2 |
| PAH2 | agggccaaggtattgtggca | 4 |
| PAH3 | gggccaaggtattgtggcag | 6 |
| PAH4 | ggccaaggtattgtggcagc | 8 |
| PAH5 | gccaaggtattgtggcagca | 10 |
| PAH6 | ccaaggtattgtggcagcaa | 12 |

In some embodiments, a gRNA comprises two or more of domains (1) and (2) and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from S. pyogenes (see, e.g., Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E. “Complete genome sequence of an M1 strain of Streptococcus pyogenes.” Proc. Natl. Acad. Sci. U.S.A. 98:4658-4663 (2001); Deltcheva E., Chylinski K., Sharma C. M., Gonzales K., Chao Y., Pirzada Z. A., Eckert M. R., Vogel J., Charpentier E. “CRISPR RNA maturation by trans-encoded small RNA and host factor RNase III.” Nature 471:602-607 (2011); and Jinek M., Chylinski K., Fonfara I., Hauer M., Doudna J. A., Charpentier E. “A programmable dual-RNA-guided DNA endonuclease in adaptive bacterial immunity.” Science 337:816-821 (2012); the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al., “Multiplex genome engineering using CRISPR/Cas systems.” Science 339:819-823 (2013); Mali, P. et al. “RNA-guided human genome engineering via Cas9.” Science 339:823-826 (2013); Hwang, W. Y. et al. “Efficient genome editing in zebrafish using a CRISPR-Cas system.” Nature Biotechnology 31:227-229 (2013); Jinek, M. et al. “RNA-programmed genome editing in human cells.” eLife 2:e00471 (2013); Dicarlo, J. E. et al. “Genome engineering in Saccharomyces cerevisiae using CRISPR-Cas systems.” Nucleic Acids Research 41:4336-4343 (2013); Jiang, W. et al. “RNA-guided editing of bacterial genomes using CRISPR-Cas systems.” Nature Biotechnology 31:233-239 (2013); the entire contents of each of which are incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a fusion protein comprising a deaminase, (e.g., a dCas9-adenosine deaminase fusion protein provided herein).

The terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

In certain embodiments, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a CRISPR enzyme in combination with (and optionally complexed with) a gRNA is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism.

Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6:1149-1154 (1988); Vigne, Restorative Neurology and Neuroscience 8:35-36(1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994). Methods of non-viral delivery of nucleic acids include lipofection, nucleofection, microinjection, biolistics, virosomes, liposomes, immunoliposomes, polycation or lipid-nucleic acid conjugates, lipid nanoparticles, artificial virions, virus-like particles, naked DNA, and agent-enhanced uptake of DNA. Lipofection is described in e.g., U.S. Pat. Nos. 5,049,386, 4,946,787, and 4,897,355, and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g., in vitro or ex vivo administration) or target tissues (e.g., in vivo administration).

Methods to deliver gene editing agents in vivo as ribonucleoproteins is another approach and provides safety advantages over nucleic acid delivery approaches. Engineered DNA-free virus-like particles (eVLPs) have been developed that efficiently package and deliver base editor or Cas9 ribonucleoproteins. By engineering VLPs to overcome cargo packaging, release, and localization bottlenecks, fourth-generation eVLPs have been developed that mediate efficient base editing in several primary mouse and human cell types. Using different glycoproteins in eVLPs alters their cellular tropism. Single injections of eVLPs into mice support therapeutic levels of base editing in multiple tissues, reducing serum Pcsk9 levels 78% following 63% liver editing, and partially restoring visual function in a mouse model of genetic blindness. In vitro and in vivo off-target editing from eVLPs was virtually undetected, an improvement over AAV or plasmid delivery. Thus, eVLPs provide promising vehicles for therapeutic macromolecule delivery that combine key advantages of both viral and nonviral delivery. See Banskota et al. Cell 185:250-265 (2021).

The preparation of lipid nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). Other lipid nanoparticle formulations are disclosed in U.S. Pat. Nos. 11,066,355; 11,059,807; US patent publications 2021/0106538 and 2021/0113466.

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retrovirus, lentivirus, adenovirus, adeno-associated virus, and herpes simplex virus vectors for gene transfer. Integration in the host genome is possible with the retrovirus, lentivirus, and adeno-associated virus gene transfer methods, often resulting in long-term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue.

Retroviral vectors comprise cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Several different AAV serotypes have been used to advantage for transduction of mammalian cells; these include, for example AAV1, AAV2, AAV4, AAV5, AAV6, AAV7, AAV8, and AAV9 that have different tropisms for cell types of interest. Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). In certain preferred embodiments, the viral vector is a split AA8 vector or a split AAV9 vector.

Packaging cells are typically used to form virus particles that are capable of infecting a host cell. Such cells include HEK 293 cells, which package adenovirus, and y2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line.

For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo, or in vitro. In some embodiments, the method comprises sampling a cell or population of cells from a human or non-human animal and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing an adenine base editor (ABE) CRISPR complex to bind to the target polynucleotide to effect correction of a mutation in said target polynucleotide thereby modifying the target polynucleotide, wherein the CRISPR complex comprises the ABE CRISPR enzyme complexed with a gRNA hybridized to a target sequence within said target polynucleotide.

In one aspect, the invention provides kits containing any one or more of the elements disclosed in the above methods and compositions. In some embodiments, the kit comprises a vector system or components for an alternative delivery system such as those described above and instructions for using the kit. In some embodiments, the vector or delivery system comprises an ABE CRISPR enzyme complexed with a gRNA for base editing of a target nucleic acid.

The kit can contain a lipid nanoparticle formulation encapsulating the appropriate base editor and at least one gRNA. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a gRNA sequence for insertion into a vector so as to operably link the gRNA sequence and a regulatory element. In some embodiments, the kit comprises a homologous recombination template polynucleotide.

In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types in methods of gene therapy.

As used herein, the term “metabolic gene” is defined as an inherited single gene anomaly, i.e., a single gene coding for an enzyme is defective, and that defect causes an enzyme deficiency. The enzyme deficiency produces an inherited metabolic disease or disorder, of which a subtype is an inborn error of metabolism. Most single gene anomalies are autosomal recessive, i.e., two defective copies of the gene must be present for the disease or trait to develop. Non-limiting examples of metabolic disorders include glucose metabolism disorders, lipid metabolism disorders, malabsorption syndromes, metabolic brain diseases, calcium metabolism disorders, DNA repair-deficiency disorders, hyperlactatemia, iron metabolism disorders, metabolic syndrome X, inborn errors of metabolism, phosphorus metabolism disorders, and acid-base imbalance. Inherited metabolic diseases previously were classified as disorders of carbohydrate metabolism, amino acid metabolism, organic acid metabolism, or lysosomal storage diseases; however, new inherited disorders of metabolism have been discovered, and the categories have multiplied. Certain major classes of congenital metabolic diseases include disorders of carbohydrate metabolism, e.g., glycogen storage disease, glucose-6-phosphate dehydrogenase (G6PD) deficiency (resulting from a mutation in the G6PD gene); disorders of amino acid metabolism, e.g., phenylketonuria, maple syrup urine disease, glutaric acidemia type 1; urea cycle disorder (urea cycle defects), e.g., carbamoyl phosphate synthetase I deficiency; disorders of organic acid metabolism (organic acidurias), e.g., alcaptonuria, 2-hydroxyglutaric acidurias; disorders of fatty acid oxidation and mitochondrial metabolism; e.g., medium-chain acyl-coenzyme A dehydrogenase deficiency (often called “MCADD”) (caused by mutations in the ACADM gene, which results in medium-chain fatty acids not being metabolized properly and leads to lethargy and hypoglycemia); disorders of porphyrin metabolism, e.g., acute intermittent porphyria; disorders of purine or pyrimidine metabolism, e.g., Lesch-Nyhan syndrome (caused by mutations in the hypoxanthine phosphoribosyltransferase 1 [HPRT1] gene and inherited in an X-linked recessive manner); disorders of steroid metabolism, e.g., lipoid congenital adrenal hyperplasia, congenital adrenal hyperplasia; disorders of mitochondrial function, e.g., Keams-Sayre syndrome; disorders of peroxisomal function, e.g., Zellweger syndrome (caused by mutations in genes encoding peroxins, e.g., PEX1, PEX2, PEX3, PEX5, PEX6, PEX10, PEX12, PEX13, PEX14, PEX16, PEX19, or PEX26 genes); and lysosomal storage disorders, e.g., Gaucher's disease (of which there are three subtypes, all of which are autosomal recessive) and Niemann-Pick disease (has an autosomal recessive inheritance pattern; Niemann-Pick types A and B are caused by a mutation in the Sphingomyelin phosphodiesterase 1 [SMPD1] gene; mutations in NPC1 gene or NPC2 gene cause Niemann-Pick disease, type C [NPC], which affects a protein used to transport lipids; Niemann-Pick type D shares a specific mutation in the NPC1 gene, patients having type D share a common Nova Scotian ancestry).

In certain aspects, an adenine base editor (ABE) complex for programming conversion of adenine to guanine in a patient in need thereof is provided where the patient has a target DNA molecule harboring a mutation associated with phenylketonuria. An exemplary ABE complex includes a modified TadA enzyme, a catalytically impaired Cas9 protein and at least one single guide RNA (sgRNA) which directs said ABE complex to said mutated target DNA molecule, which upon contact converts adenosine in said mutation to inosine, thereby catalyzing an A-T to G-C transition following DNA repair or DNA replication.

The activity window of each ABE typically ranges across several positions within the protospacer DNA sequence (e.g., the ABE8.8 window ranges from position 3 to position 9, with peak editing observed at position 6 of the protospacer), with different ABEs having different windows (Anzalone et al., 2020). ABEs have the potential to edit any adenine within the window, which could include a desired target adenine but also undesired additional adenines (bystander edits). Published ABEs with Streptococcus pyogenes Cas9 nickase include so-called eighth-generation ABEs (harboring optimized deaminase domains resulting from eight rounds of molecular evolution)—the most commonly used to date are ABE8.8, ABE8.20, and ABE8e—and circularly permuted or inlaid ABEs, in which the deaminase domain is embedded within a loop of the Cas9 nickase protein, rather than fused to the N-terminal end, which has the effect of shifting the editing window further towards the 3′ end of the protospacer sequence (Gaudelli et al., 2020; Richter et al., 2020; Chu et al., 2021). Similar ABEs with Cas9 nickase from other bacterial species (e.g., Staphylococcus aureus) have been reported (Gaudelli et al., 2020; Richter et al., 2020).

### Materials and Methods

The following materials and methods are provided to facilitate the practice of the present invention.

For prime editing, the pCMV-PEmax-P2A-hMLHldn plasmid (Addgene #174828) was used to express the prime editor (PEmax), the pU6-tevopreq1-GG-acceptor plasmid (Addgene #174038) was used to express the prime editing guide RNA (pegRNA)—for insertion of the PAH c.1222C>T variant—following Gibson cloning of the oligonucleotide-synthesized pegRNA sequence, and the pGuide plasmid (Addgene #64711) was used to express the nicking guide RNA (ngRNA) following subcloning of the oligonucleotide-synthesized ngRNA sequence. For base editing, a variety of adenine base editor (ABE)-expressing plasmids were used including: SpG-ABE8e (Addgene #185911), SpG-ABE8.20 (Addgene #185916), SpRY-ABE8e (Addgene #185912), SpRY-ABE8.20 (Addgene #185917), and SpRY-ABE8.8 (made by combining elements of Addgene #185912 and Addgene #136294). The pGuide plasmid (Addgene #64711) was used to express each accompanying guide RNA (specific for the PAH c.1222C>T variant) following subcloning of the oligonucleotide-synthesized gRNA sequence.

100-mer PAH4 gRNA was chemically synthesized under solid phase synthesis conditions by a commercial supplier (Agilent) with end-modifications as well as heavy 2′-O-methylribosugar modification:

(SEQ ID NO: 19)
PAH4,
5′-mG*mG*mC*CAAGGUAUUGUGGCAGCGUUUUAGAmGmCmUmAmGm
AmAmAmUmAmGmCAAGUUAAAAUAAGGCUAGUCCGUUAUCAmAmCmUm
UmGmAmAmAmAmAmGmUmGmGmCmAmCmCmGmAmGmUmCmGmGmUmGm
CmU*mU*mU*mU-3′;

where “m” and * respectively indicate 2′-O-methylation and phosphorothioate linkage. SpRY-ABE8.8 mRNA was produced via in vitro transcription (IVT) and purification. In brief, a plasmid DNA template containing a codon-optimized SpRY-ABE8.8 coding sequence to express the SpRY-ABE8.8 protein (SEQ ID NO: 20) and a 3′ polyadenylate sequence was linearized. An IVT reaction containing linearized DNA template, T7 RNA polymerase, NTPs, and cap analog was performed to produce mRNA containing N1-methylpseudouridine. After digestion of the DNA template with DNase I, the mRNA product underwent purification and buffer exchange, and the purity of the final mRNA product was assessed with spectrophotometry and capillary gel electrophoresis. Elimination of double-stranded RNA contaminants was assessed using dot blots and transfection into human dendritic cells. Endotoxin content was measured using a chromogenic Limulus amebocyte lysate (LAL) assay; all assays were negative.

Lipid nanoparticles (LNPs) were formulated as previously described, with the lipid components (SM-102, 1,2-distearoyl-sn-glycero-3-phosphocholine, cholesterol, and a PEG-lipid) being rapidly mixed with an aqueous buffer solution containing ABE8.8 mRNA and PAH4 gRNA in a 1:1 ratio by weight in 25 mM sodium acetate (pH 4.0). The resulting LNP formulations were subsequently dialyzed against sucrose-containing buffer, concentrated using Amicon Ultra-15 mL Centrifugal Filter Units (Millipore Sigma), sterile-filtered using 0.2-μm filters, and frozen until use.

HuH-7 human hepatoma cells were obtained from the Japanese Collection of Research Bioresources (JCRB) Cell Bank and maintained in culture with DMEM containing 1 g/L glucose and supplemented with 10% FBS (Thermo Fisher). HuH-7 cells were maintained in Dulbecco's modified Eagle's medium (containing 4 mM L-glutamine and 1 g/L glucose) with 10% fetal bovine serum and 1% penicillin/streptomycin at 37° C. with 5% CO₂. To make homozygous R408W HuH-7 clones, HuH-7 cells were seeded on 6-well plates (Corning) at 3.5×10⁵ cells per well. At 16-24 hours after seeding, cells were transfected at approximately 80-90% confluency with 9 μL TransIT®-LT1 Transfection Reagent, 1.5 μg PEmax plasmid, 0.75 μg epegRNA-expressing plasmid, and 0.75 μg nicking gRNA plasmid. Cells were dissociated with trypsin 48 hours post-transfection and replated onto 10-cm plates (5,000 cells/plate) with conditioned medium to facilitate recovery, and genomic DNA was isolated from the remainder of the cells as a pool to perform PCR and Sanger sequencing of the PAH locus at the site of the c.1222C>T variant. Single cells were permitted to expand for 7-14 days to establish clonal populations. Colonies were manually picked and replated into individual wells of a 96-well plate. Genomic DNA was isolated from individual clones, and PCR and Sanger sequencing was performed to identify homozygous R408W HuH-7 clones. One representative clone was expanded for use in subsequent studies.

For assessment of base editing of the PAH c.1222C>T variant, homozygous R408W HuH-7 cells were seeded on 6-well plates (Corning) at 3.5×10⁵ cells per well. At 16-24 hours after seeding, cells were transfected at approximately 80-90% confluency with 9 μL TransIT®-LT1 Transfection Reagent (MIR2300, Mirus), 2 μg base editor plasmid, and 1 μg gRNA plasmid per well according to the manufacturer's instructions. In other experiments, LNPs were added at various doses (quantified by the total amount of RNA within the LNPs) directly to the media. Cells were cultured for 72 hours after transfection, and then media were removed, cells were washed with 1×DPBS (Corning), and genomic DNA was isolated using the DNeasy Blood and Tissue Kit (QIAGEN) according to the manufacturer's instructions.

PCR amplification of the target sequences (PAH locus at the site of the c.1222C>T variant) in genomic DNA samples from transfected HuH-7 cells was performed using NEBnext High-Fidelity 2×PCR Master Mix (New England Biolabs) with locus-specific primers containing 5′ Nextera adaptor sequences (Illumina), followed by purification of the PCR amplicons with the NGS Normalization 96-Well Kit (Norgen Biotek). A second round of PCR with the Nextera XT Index Kit V2 Set A and/or Nextera XT Index Kit V2 Set D (Illumina), followed by purification with the NGS Normalization 96-Well Kit, generated barcoded libraries, which were pooled and quantified using a Qubit 3.0 Fluorometer. After denaturation, dilution to 10 pM, and supplementation with 15% PhiX, the pooled libraries underwent single-end or paired-end next-generation sequencing on an Illumina MiSeq System. The amplicon sequencing data were analyzed with CRISPResso2 v2 (available on the world wide web at: crispresso.pinellolab.partners.org/) and custom scripts. For on-target editing, A-to-G editing was quantified at the site of the R408W variant (position 5 of the PAH4 protospacer sequence) and at the sites of potential bystander adenine editing (positions 6 and 10 of the PAH4 protospacer sequence). In some cases, PCR amplicons were subjected to confirmatory Sanger sequencing, performed by GENEWIZ, with editing frequencies estimated from the chromatograms.

A PKU mouse model with one or more humanized PAH R408W alleles was generated using in vitro transcribed Cas9 mRNA, a synthetic gRNA (spacer sequence 5′-AGCGAACGGAGAAGGGCCGG-3′ (SEQ ID NO: 21)) (Integrated DNA Technologies), and a synthetic single-strand DNA oligonucleotide (Integrated DNA Technologies) with homology arms matching the target site and harboring the R408W variant and synonymous variants (bold with underline): 5′-AAAAGCCACTTGGAACTCCTCCAGGATAACCTGTCTTTAAATGGTGTCCTTCACTG GGGTCCTTGGTTTTGGTTTCAGGAACTTTGCTGCCACAATACCTTGGCCCTTCTCAGT TCGCTACGACCCCTACACTCAAAGGGTTGAGGTCCTGGACAATACTCAGCAGTTGAA GATTTTAGCTGACTCCATTAATAGTAAGT-3′ (SEQ ID NO: 22). The mixture of the 3 components was injected into cytoplasm of fertilized oocytes from C57BL/6J mice at the Penn Vet Transgenic Mouse Core (available on the world wide web at: vet.upenn.edu/research/core-resources-facilities/transgenic-mouse-core). Genomic DNA samples from founders were screened for knock-in of the desired sequence in the Pah locus via homology-directed repair. Founders with the humanized R408W allele were bred through two generations to obtain homozygous mice.

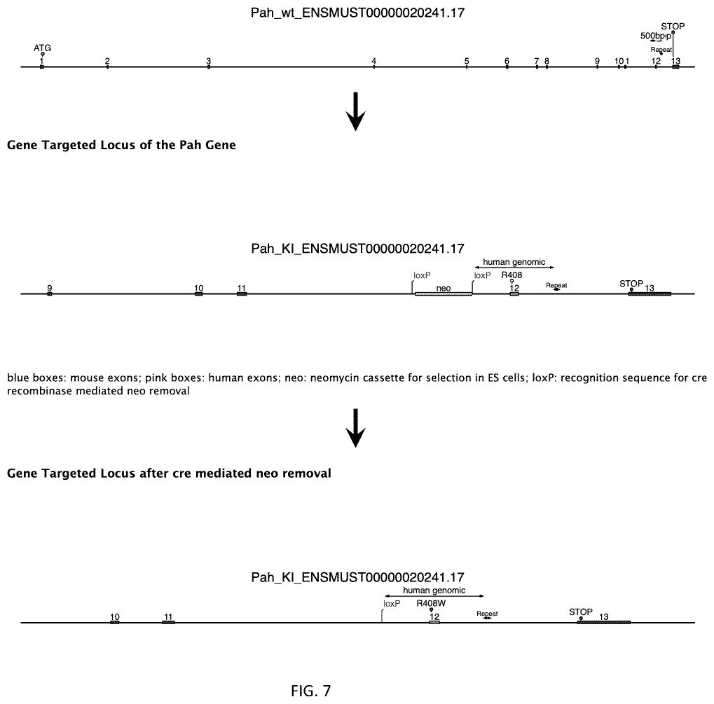

A different PKU mouse model with one or more humanized PAH R408W alleles was generated through the use of homologous recombination in mouse embryonic stem cells, followed by blastocyst injections, generation of chimeras, and subsequent breeding, as illustrated in FIG. 7.

Homozygous and compound heterozygous humanized PKU mice, as well as heterozygous humanized non-PKU mice, were generated as littermates/colony-mates via timed breeding, in some cases using wild-type C57BL/6J mice (stock no. 000664) obtained from The Jackson Laboratory. Genotyping was performed using PCR amplification from genomic DNA samples (prepared from clipped tails/ears) followed by next-generation sequencing. LNPs were administered to the mice via retro-orbital injection under anesthesia with 1%-2% inhaled isoflurane. In short-term studies, mice were euthanized at 1 week after treatment, and 8 liver samples (2 from each lobe) and samples of other organs were obtained on necropsy and processed with the DNeasy Blood and Tissue Kit (QIAGEN) as per the manufacturer's instructions to isolate genomic DNA. Next-generation sequencing results from the liver samples were averaged to provide quantification of whole-liver editing. Blood samples were collected via the tail tip at various time points (pre-treatment, day 1, day 2, day 3, and day 7), in the early afternoon to account for diurnal variation in blood phenylalanine levels.

The blood phenylalanine levels were measured by an enzymatic method using the Phenylalanine Assay Kit (MAK005, Millipore Sigma) according to the manufacturers' instructions. Briefly, plasma samples were deproteinized with a 10 kDa MWCO spin filter (CLS431478-25EA, Millipore Sigma) and pre-treated with 5 μL of tyrosinase for 10 minutes at room temperature prior to start of the assay. Reaction mixes were made according to the manufacturers' instructions, and the fluorescence intensity of each sample was measured (λex=535/λem=587 nm).

Mice were euthanized by CO₂ inhalation at the time of tissue collection. For next-generation sequencing (NGS), PCR reactions were performed using NEBNext Polymerase (NEB) with locus-specific primers containing 5′ Nextera adaptor sequences (Illumina). The following program was used for all genomic DNA PCRs: 98° C. for 20 seconds, 35×(98° C. for 20 seconds, 57° C. for 30 seconds, 72° C. for 10 seconds), 72° C. for 2 minutes. PCR products were visualized via capillary electrophoresis (QIAxcel, QIAGEN) and then purified and normalized via the NGS Normalization 96-Well Kit (Norgen Biotek Corporation). A secondary barcoding PCR was conducted to add Illumina barcodes (Nextera XT Index Kit V2 Set A and/or Nextera XT Index Kit V2 Set D) using ≈15 ng of first-round PCR product as template, followed by purification and normalization. Final pooled libraries were quantified using a Qubit 3.0 Fluorometer (Thermo Fisher Scientific) and then after denaturation, dilution to 10 pM, and supplementation with 15% PhiX, underwent single-end or paired-end sequencing on an Illumina MiSeq System. The amplicon sequencing data were analyzed with CRISPResso2 v2 and custom scripts to quantify editing. For on-target editing, A-to-G editing was quantified at the site of the R408W variant (position 5 of the PAH4 protospacer sequence) and at the sites of potential bystander adenine editing (positions 6 and 10 of the PAH4 protospacer sequence).

The following examples are provided to illustrate certain embodiments of the invention. They are not intended to limit the invention in any way.

### Example 1

Meta Analysis of PKU Patients with PAH c.1222C>T Variants and Screening ABEs for Corrective Activity

Gene-editing methods and compositions include CRISPR-Cas9 and -Cas12 nucleases (Jinek et al., 2012; Zetsche et al., 2015; Strecker et al., 2019), CRISPR cytosine base editors (Komor et al., 2016), CRISPR adenine base editors (Gaudelli et al., 2017), and CRISPR prime editors (Anzalone et al., 2019).

CRISPR base editors are an attractive gene-editing modality because they function efficiently for introducing precise targeted alterations without the need for double-strand breaks, in contrast to CRISPR-Cas9 and other gene-editing nucleases (FIG. 1A, FIG. 1B). Adenine base editors (ABEs) can induce targeted A→G edits in DNA (T→C on the opposing strand). Each ABE uses its core Cas9 nickase protein with a guide RNA (gRNA) to engage a double-strand protospacer DNA sequence, which is flanked by a protospacer-adjacent motif (PAM) sequence on its 3′ end. Some ABEs use a core Cas9 nickase protein that has been engineered to accommodate a variety of PAM sequences, making it much more flexible with respect to target site selection. For example, standard S. pyogenes Cas9 has a PAM preference of NGG, but SpG-Cas9 has a relaxed PAM preference of NGN, and SpRY-Cas9 is “near-PAMless” and can accommodate almost any PAM, while still using a standard S. pyogenes gRNA (Walton et al., 2020). Other engineered core Cas9 nickase proteins have a highly specific PAM preference for a sequence other than NGG (e.g., NGA, NGCG, NGCA, NAA, or NAAG).

Unlike Cas9 and Cas12, ABEs do not make double-strand breaks and have minimal risk of inducing large deletions, chromosomal abnormalities, and chromothripsis (shattering). Instead, each ABE uses an evolved deoxyadenosine deaminase domain—typically fused to the N-terminal end of the Cas9 nickase—to chemically modify an adenosine nucleoside on one DNA strand, which (in combination with nicking of the other strand) enables highly precise and efficient A→G transition mutations at the targeted site.

Phenylketonuria (PKU) is an autosomal recessive disorder caused by mutations in the gene encoding phenylalanine hydroxylase (PAH), resulting in the accumulation of phenylalanine (Phe) to neurotoxic levels due to the inability to convert Phe into tyrosine. Untreated PKU can manifest high blood Phe levels of >1200 μmol/L. However, current treatment guidelines state that blood Phe levels should ideally be maintained in the range of 120-360 μmol/L (Vockley et al., 2014). The existing treatment options range from a strict low-Phe diet to an oral medication (sapropterin, a cofactor of PAH) to an injectable enzyme substitution therapy (pegvaliase). All are chronic, everyday interventions to which patients must adhere for the lifetime if they are to prevent cognitive impairment and a range of neuropsychiatric complications. The burden of intensive Phe control and monitoring faced by PKU patients and their families is profound, especially with treatments that can be unpalatable and cost-prohibitive. Lack of adherence to the guidelines on the part of many patients with PKU is not only a serious risk, but also common and therefore expected. Studies have shown that more than 70% of adults with PKU are noncompliant with therapy (Jurecki et al., 2017). As such, durable and, ideally, curative therapies would be needed to optimally address the medical needs of PKU patients.

Out of the more than 1,000 PAH variants that have been cataloged in patients (Regier and Greene, 2017), the most common pathogenic variants linked to classic PKU are all transition mutations, specifically G→A or C→T variants on the sense or antisense strand (Hillert et al., 2020). As such, each of these variants is potentially amenable to correction by gene editing. For example, adenine base editing, which can engineer site-specific A→G changes on either DNA strand (Gaudelli et al., 2017), would be effective to correct each of these variants. The PAH c.1222C>T (p.Arg408Trp, R408W) variant is by far the most common pathogenic variant for PKU worldwide. The PAH c.1222C>T variant has its highest prevalence in European populations (e.g., present in 98.9% of PKU patients in Estonia, 89.2% in Poland, 75.7% in Russia, 43.6% in Sweden, and 35.7% in Germany), Australia (34.7%), and the United States (32.9%) (Hillert et al., 2020).

Patients homozygous for the PAH c.1222C>T variant have the most severe form of PKU and typically do not respond to sapropterin (Leuders et al., 2014), limiting their treatment options. Patients with the R408W variant do respond to pegvaliase, a once-daily injected bacterial-derived enzyme that directly catabolizes Phe, albeit with a substantial risk of anaphylaxis. However, in clinical trials, PKU patients on pegvaliase had only a mean Phe reduction of 51% at one year after initiation (from 1233 μmol/L to 565 μmol/L), meaning that a large proportion of the patients did not achieve the guideline-directed Phe goal of <360 μmol/L (Burton et al., 2020).

To define the degree of adherence to Phe treatment and monitoring goals by PKU patients with the PAH R408W variant, real-world data from a PKU cohort managed at a metabolic specialty clinic at an academic medical center were analyzed. Among 129 patients with PKU followed by the Children's Hospital of Philadelphia (CHOP) Metabolic Clinic, 32 (24.8%) were found to be compound heterozygous for the PAH R408W allele, while 4 (3.1%) were homozygous for the variant. This may be an underestimate of true R408W prevalence, as genotype information was not available for some older patients with PKU.

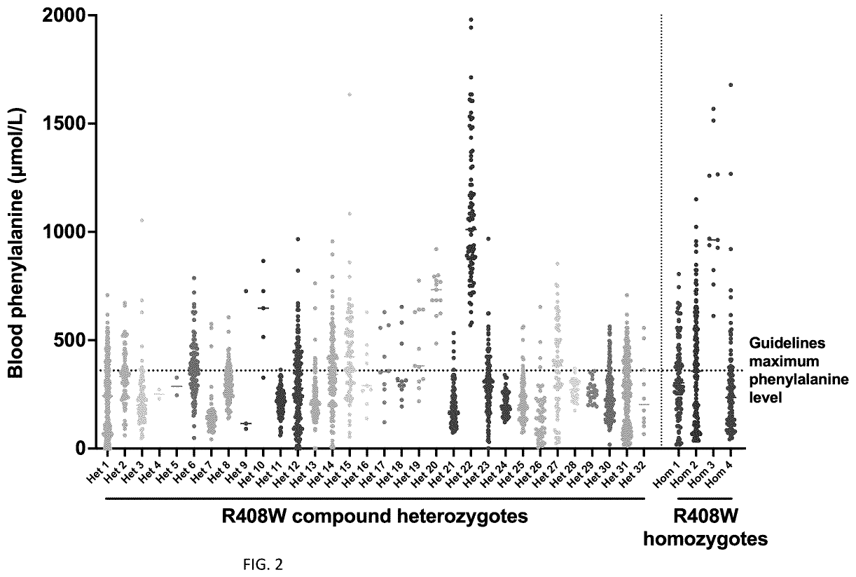

Consensus management guidelines from the American College of Medical Genetics recommend patients with PKU maintain Phe levels in the 120-360 μmol/L range (Vockley et al., 2014). Levels above 600 μmol/L can be neurotoxic and are associated with poor psychiatric and neurocognitive outcomes (Romani et al., 2019; Ashe et al., 2019; Thomas et al., 2023). In the study cohort, 33 of 36 (91.6%) treated patients had at least a single Phe level above 360 μmol/L, and 25 of 36 (69.4%) had at least a single level above 600 μmol/L (FIG. 2). Furthermore, 10 of 36 (27.7%) had lifetime average Phe levels above 360 μmol/L. The four patients homozygous for the R408W allele demonstrated especially poor metabolic control.

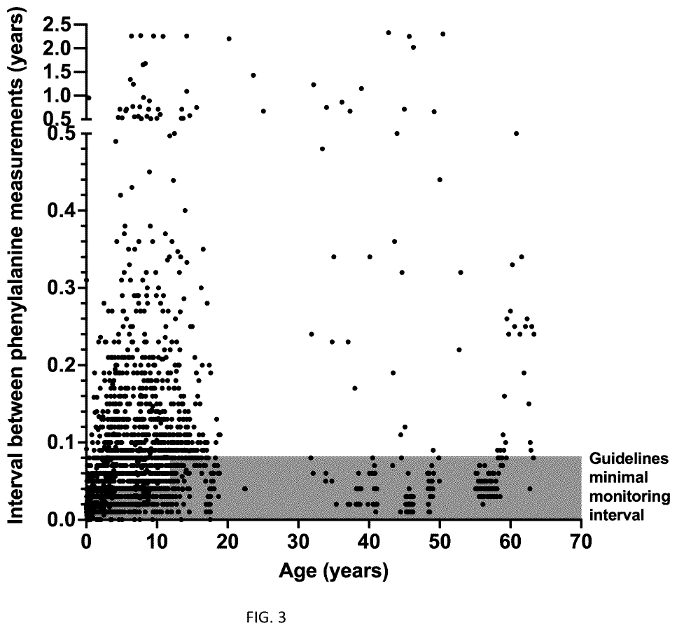

In addition to high Phe levels, patients with R408W variants demonstrated poor adherence to Phe monitoring schedules. While exact recommended intervals can vary by clinical scenario, general guidelines include weekly Phe monitoring prior to age 1 year, every-2-week monitoring for ages 1-12 years, and every-2-4 week monitoring in adolescents and adults (FIG. 3, grey box) (Vockley et al., 2014). The interval since the last recorded value for every Phe measurement in the cohort was calculated (FIG. 3, black dots). The interval between monitoring labs was longer than recommended for 1,556 of 2,535 (61.3%) of the Phe values measured. Collectively, these data demonstrate that most patients with R408W variants have poor metabolic control and are at risk for chronic neurologic damage, providing a strong rationale for a one-time, definitive gene editing treatment to correct the PAH R408W variant in the liver.

Studies have been performed demonstrating the viability of adenine base editing as a therapeutic approach in vivo. Most notably, lipid nanoparticles (LNPs) have proven effective at delivering an adenine base editor, encoded in mRNA, into the livers of non-human primates (Musunuru et al., 2021; Rothgangl et al., 2021). An adenine base editor efficiently introduced a loss-of-function variant into the PCSK9 cholesterol-regulating gene, achieving saturation editing of the hepatocytes in the liver and reducing the PCSK9 protein by ≈90% without any adverse health consequences (Musunuru et al., 2021). In a recent clinical trial, LNP-mediated delivery of a nuclease editor (CRISPR-Cas9) into the liver to introduce loss-of-function mutations into a target gene (TTR) was safely tolerated and resulted in up to 96% reduction of the protein product (transthyretin) (Gillmore et al., 2021). Accordingly, a broad range of editing therapies can now be developed to ameliorate symptoms of a variety of diseases for which gene alterations in the liver would be curative.

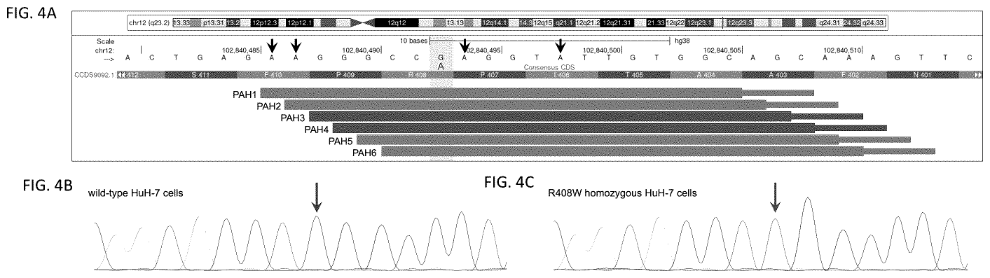

With PCSK9, the goal was to edit the wild-type gene, which is endogenous in primary human hepatocytes or cultured hepatocyte lines from any source. In contrast, with PAH, the goal is to correct a rare human mutation, c.1222C>T (FIG. 4). However, there are no readily available primary human hepatocytes or cultured hepatocyte lines bearing that variant. Moreover, due to the limited ability of primary human hepatocytes to proliferate or to persist in culture more than a few days, there is no possibility of editing the variant into the hepatocytes to allow for subsequent testing of variant correction. Accordingly, prime editing was used to introduce the PAH R408W variant into HuH-7 human hepatoma cells, which have the advantage of being hepatocyte-like cells that proliferate indefinitely in culture, are highly transfectable, and can undergo single-cell cloning to generate genetically modified cell lines. The initial attempt at the use of a prime editor (PEmax) with an engineered pegRNA and a ngRNA achieved 34% insertion of the R408W variant into bulk HuH-7 cells when delivered via plasmid transfection. Single-cell cloning to expand edited HuH-7 cell lines yielded lines that were homozygous for the R408W variant (FIG. 4). A representative homozygous R408W HuH-7 cell line was used for further experiments.

Developed by directed evolution of a seventh-generation ABE, ABE7.10, at least forty-one modified eighth-generation ABEs have been reported that all can have higher editing efficiencies compared to ABE7.10 in mammalian cells (Gaudelli et al., 2020; Richter et al., 2020). In addition, 30 inlaid base editors (IBEs) have been reported, several of which have higher editing efficiencies compared to the standard N-terminal deaminase-fused ABE, while having 3′-shifted editing windows (Chu et al., 2021). Of note, the possibility of using a SaCas9-containing ABE was considered. However, there is no SaCas9 NNGRRT PAM or SaCas9 KKH variant NNNRRT PAM that is optimally positioned to place the target PAH c.1222C>T adenine within the editing window in such a way as to avoid or minimize counterproductive bystander editing, due to the much broader editing windows with SaCas9-containing ABEs compared to SpCas9-containing ABEs. As such, SpCas9-containing ABEs were utilized.

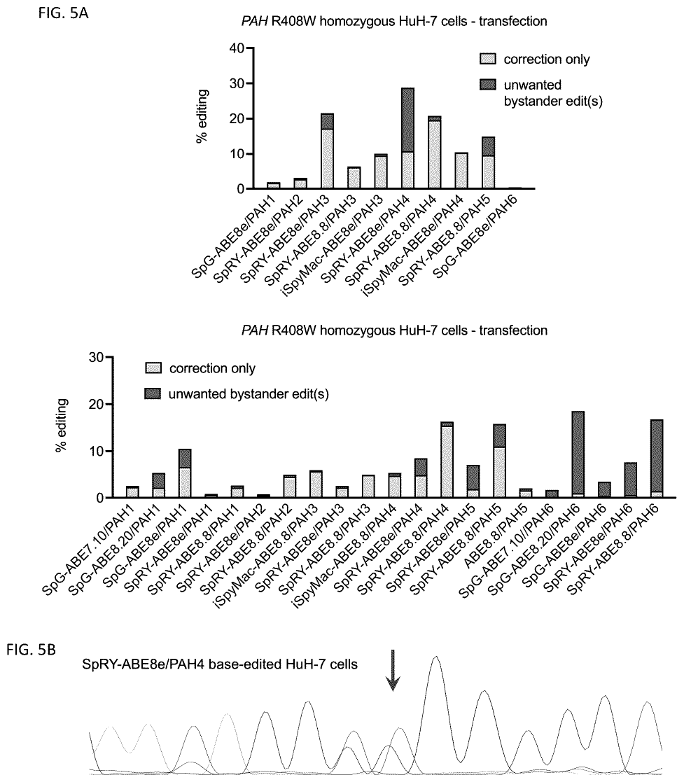

Using the clonal HuH-7 cell line homozygous for the PAH c.1222C>T variant (homozygous R408W HuH-7 cells), generated with prime editing as described above, a variety of eighth-generation ABEs in combination with some or all of six gRNAs (SEQ ID NOS: 1, 3, 5, 7, 9, and 11) (FIG. 4) were tested for their ability to correct the variant (FIG. 5). To expand the set of potential target sites beyond those that can be targeted by standard SpCas9 with NGG PAMs, ABEs with core SpG-Cas9 (NGN PAMs), SpRY-Cas9 (near-PAMless), and iSpyMacCas9 (NAA PAM) nickases were tested. All of the ABE/gRNA sets corrected some proportion of the variant alleles to wild-type, but with highly varied levels of editing of the PAH c.1222C>T variant and highly varied levels of bystander editing. Of note, bystander editing of the adenine in the position adjacent to the position of the c.1222C>T (c.1221) would represent a synonymous edit not expected to be of any functional consequence. Among the ABE/gRNA sets tested, SpRY-ABE8.8 (SEQ ID NO: 20) in combination with the PAH4 gRNA (SEQ ID NO: 7) displayed the most favorable balance of efficient variant correction and minimized bystander editing at positions other than c.1221.

### Example 2

### ABE Activity in Human Hepatocytes Via LNP Delivery.

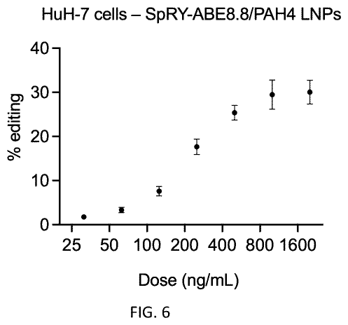

For LNP delivery, the PAH4 gRNA with appropriately positioned 2′-O-methyl and phosphorothioate modifications was synthesized by Agilent. mRNA encoding SpRY-ABE8.8 was generated via in vitro transcription and purification by the University of Pennsylvania Engineered mRNA and Targeted Nanomedicine Core. The Core also formulated the mRNA with the gRNA into LNPs containing standard lipid components (ionizable cationic lipid, 1,2-distearoyl-sn-glycero-3-phosphocholine, cholesterol, and a PEG-lipid). We performed dose-response studies with the LNPs using the homozygous R408W HuH-7 cell line (FIG. 6). These studies showed that there was =30% corrective editing at higher doses.

### Example 3

Generating Two Humanized Mouse Models with the Human PAH c.1222C>T Variant.

In order to assess for editing activity of the prioritized ABE/gRNA set(s) in hepatocytes in vivo, an animal model that harbors not only the PAH c.1222C>T variant but also the protospacer DNA sequence and the surrounding sequence context that allows for a functional readout of variant correction must be generated. Accordingly, humanized mouse models in which a portion of the endogenous mouse PAH locus has been replaced with the orthologous portion of the human PAH locus containing the variant have been created. This degree of humanization facilitates assessment of the therapeutic effect of base editing of the PAH c.1222C>T variant via disease-relevant phenotypic readouts.

A humanized mouse model can be generated using a number of approaches. In one approach, the mouse exon containing the site of the PAH c.1222C>T variant (exon 12) was replaced as well as =500 base pairs of the flanking introns (intron 11 and intron 12) (FIG. 7). This was achieved via electroporation of a PAH targeting vector into mouse embryonic stem cells and generation of chimeras, followed by breeding, in the inbred C57BL/6J background. Homozygous mice, when maintained on a normal chow diet, displayed signs of PKU including elevated blood Phe levels and hypopigmentation (agouti instead of black fur). The humanized PAH c.1222C>T variant can be maintained in the heterozygous state, with heterozygous mice being entirely healthy.

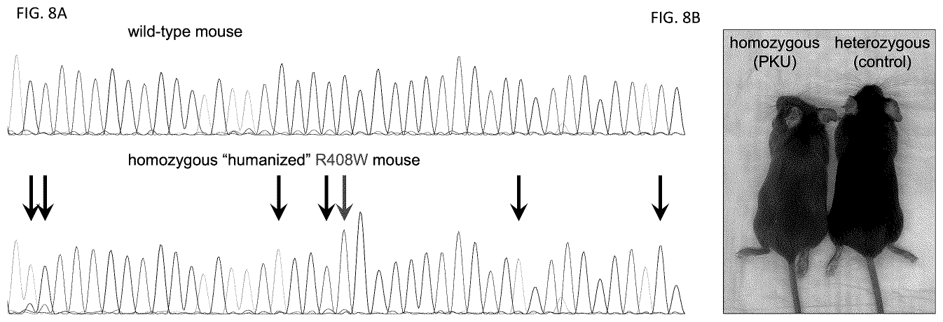

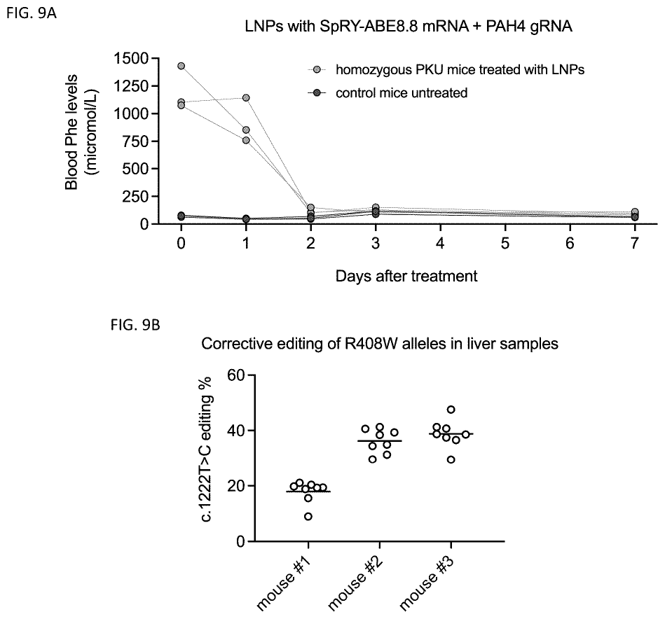

In another approach, we used CRISPR-Cas9 targeting in mouse embryos to generate a minimally humanized PKU model, in the C57BL/6J background, in which we replaced a small portion of the endogenous mouse Pah exon 12 with the orthologous human sequence spanning the protospacers of the tested gRNAs (SEQ IQ NOS: 2, 4, 6, 8, 10, and 12) and containing the c.1222C>T variant (FIG. 8A). Homozygous mice had phenotypes consistent with PKU (FIG. 8B, FIG. 9A).

### Example 4

### ABE Activity in Mice Via LNP Delivery.

For LNP delivery, the PAH4 gRNA with appropriately positioned 2′-O-methyl and phosphorothioate modifications were synthesized by Agilent. mRNA encoding SpRY-ABE8.8 ABE was generated via in vitro transcription and purification by the University of Pennsylvania Engineered mRNA and Targeted Nanomedicine Core. The Core also formulated the mRNA with the gRNA into LNPs containing standard lipid components (ionizable cationic lipid, 1,2-distearoyl-sn-glycero-3-phosphocholine, cholesterol, and a PEG-lipid).

In a short-term study, three homozygous R408W (PKU) mice (6-10 weeks of age) were treated with SpRY-ABE8.8/PAH4 LNPs, with three non-PKU colony mates serving as controls (FIG. 9A). At baseline, the PKU mice had blood phenylalanine (Phe) levels ranging from 1075-1431 μmol/L (highly elevated levels consistent with PKU), whereas the non-PKU mice had blood Phe levels <120 μmol/L (similar to human profiles). All the treated mice had largely normalized Phe levels at 48 hours after treatment (90% mean reduction). The treated mice were necropsied 1 week after treatment to assess editing in the liver. The desired corrective editing in the whole liver with SpRY-ABE8.8/PAH4 LNPs ranged from 18% to 39% (FIG. 9B).

### REFERENCES

Anzalone A V, Koblan L W, Liu D R. Genome editing with CRISPR-Cas nucleases, base editors, transposases and prime editors. Nat Biotechnol. 2020 July; 38(7):824-844.
Anzalone A V, Randolph P B, Davis J R, Sousa A A, Koblan L W, Levy J M, Chen P J, Wilson C, Newby G A, Raguram A, Liu D R. Search-and-replace genome editing without double-strand breaks or donor DNA. Nature. 2019 December; 576(7785):149-157.
Ashe K, Kelso W, Farrand S, Panetta J, Fazio T, De Jong G, Walterfang M. Psychiatric and Cognitive Aspects of Phenylketonuria: The Limitations of Diet and Promise of New Treatments. Front Psychiatry. 2019 Sep. 10; 10:561.
Banskota S, Raguram A, Suh S, Du S W, Davis J R, Choi E H, Wang X, Nielsen S C, Newby G A, Randolph P B, Osborn M J, Musunuru K, Palczewski K, Liu D R. Engineered virus-like particles for efficient in vivo delivery of therapeutic proteins. Cell. 2022 Jan. 20; 185(2):250-265.e16.
Blau N, van Spronsen F J, Levy H L. Phenylketonuria. Lancet. 2010 Oct. 23; 376(9750):1417-27.
Burton B K, Longo N, Vockley J, Grange D K, Harding C O, Decker C, Li M, Lau K, Rosen O, Larimore K, Thomas J; PAL-002 and PAL-004 Investigators. Pegvaliase for the treatment of phenylketonuria: Results of the phase 2 dose-finding studies with long-term follow-up. Mol Genet Metab. 2020 August; 130(4):239-246.
Cancellieri S, Zeng J, Lin L Y, Tognon M, Nguyen M A, Lin J, Bombieri N, Maitland S A, Ciuculescu M F, Katta V, Tsai S Q, Armant M, Wolfe S A, Giugno R, Bauer D E, Pinello L. Human genetic diversity alters off-target outcomes of therapeutic gene editing. Nat Genet. 2023 January; 55(1):34-43.
Chadwick A C, Evitt N H, Lv W, Musunuru K. Reduced Blood Lipid Levels With In Vivo CRISPR-Cas9 Base Editing of ANGPTL3. Circulation. 2018 Feb. 27; 137(9):975-977.
Chadwick A C, Wang X, Musunuru K. In Vivo Base Editing of PCSK9 (Proprotein Convertase Subtilisin/Kexin Type 9) as a Therapeutic Alternative to Genome Editing. Arterioscler Thromb Vase Biol. 2017 September; 37(9):1741-1747. doi: 10.1161/ATVBAHA.117.309881. Epub 2017 Jul. 27.
Chu S H, Packer M, Rees H, Lam D, Yu Y, Marshall J, Cheng L I, Lam D, Olins J, Ran F A, Liquori A, Gantzer B, Decker J, Born D, Barrera L, Hartigan A, Gaudelli N, Ciaramella G, Slaymaker I M. Rationally Designed Base Editors for Precise Editing of the Sickle Cell Disease Mutation. CRISPR J. 2021 April; 4(2):169-177.
Davis J R, Wang X, Witte I P, Huang T P, Levy J M, Raguram A, Banskota S, Seidah N G, Musunuru K, Liu D R. Efficient in vivo base editing via single adeno-associated viruses with size-optimized genomes encoding compact adenine base editors. Nat Biomed Eng. 2022 November; 6(11):1272-1283.
Donohoue P D, Pacesa M, Lau E, Vidal B, Irby M J, Nyer D B, Rotstein T, Banh L, Toh M S, Gibson J, Kohrs B, Baek K, Owen A L G, Slorach E M, van Overbeek M, Fuller C K, May A P, Jinek M, Cameron P. Conformational control of Cas9 by CRISPR hybrid RNA-DNA guides mitigates off-target activity in T cells. Mol Cell. 2021 Sep. 2; 81(17):3637-3649.e5.
Essalmani R, Weider E, Marcinkiewicz J, Chamberland A, Susan-Resiga D, Roubtsova A, Seidah N G, Prat A. A single domain antibody against the Cys- and His-rich domain of PCSK9 and evolocumab exhibit different inhibition mechanisms in humanized PCSK9 mice. Biol Chem. 2018 Nov. 27; 399(12):1363-1374.
Gaudelli N M, Komor A C, Rees H A, Packer M S, Badran A H, Bryson D I, Liu D R. Programmable base editing of A·T to G·C in genomic DNA without DNA cleavage. Nature. 2017 Nov. 23; 551(7681):464-471.
Gaudelli N M, Lam D K, Rees H A, Sold-Esteves N M, Barrera L A, Born D A, Edwards A, Gehrke J M, Lee S J, Liquori A J, Murray R, Packer M S, Rinaldi C, Slaymaker I M, Yen J, Young L E, Ciaramella G. Directed evolution of adenine base editors with increased activity and therapeutic application. Nat Biotechnol. 2020 July; 38(7):892-900.
Gillmore J D, Gane E, Taubel J, Kao J, Fontana M, Maitland M L, Seitzer J, O'Connell D, Walsh K R, Wood K, Phillips J, Xu Y, Amaral A, Boyd A P, Cehelsky J E, McKee M D, Schiermeier A, Harari O, Murphy A, Kyratsous C A, Zambrowicz B, Soltys R, Gutstein D E, Leonard J, Sepp-Lorenzino L, Lebwohl D. CRISPR-Cas9 In Vivo Gene Editing for Transthyretin Amyloidosis. N Engl J Med. 2021 Aug. 5; 385(6):493-502.
Grisch-Chan H M, Schwank G, Harding C O, Töany B. State-of-the-Art 2019 on Gene Therapy for Phenylketonuria. Hum Gene Ther. 2019 October; 30(10):1274-1283.
Hillert A, Anikster Y, Belanger-Quintana A, Burlina A, Burton B K, Carducci C, Chiesa A E, Christodoulou J, [symbol] or [symbol] ević M, Desviat L R, Eliyahu A, Evers R A F, Fajkusova L, Feillet F, Bonfim-Freitas P E, Gizewska M, Gundorova P, Karall D, Kneller K, Kutsev S I, Leuzzi V, Levy H L, Lichter-Konecki U, Muntau A C, Namour F, Oltarzewski M, Paras A, Perez B, Polak E, Polyakov A V, Porta F, Rohrbach M, Scholl-Bürgi S, Spdcola N, Stojiljković M, Shen N, Santana-da Silva L C, Skouma A, van Spronsen F, Stoppioni V, Thöny B, Trefz F K, Vockley J, Yu Y, Zschocke J, Hoffmann G F, Garbade S F, Blau N. The Genetic Landscape and Epidemiology of Phenylketonuria. Am J Hum Genet. 2020 Aug. 6; 107(2):234-250.
Jinek M, Chylinski K, Fonfara I, Hauer M, Doudna J A, Charpentier E. A programmable dual-RNA-guided DNA endonuclease in adaptive bacterial immunity. Science. 2012 Aug. 17; 337(6096):816-21.
Jurecki E R, Cederbaum S, Kopesky J, Perry K, Rohr F, Sanchez-Valle A, Viau K S, Sheinin M Y, Cohen-Pfeffer J L. Adherence to clinic recommendations among patients with phenylketonuria in the United States. Mol Genet Metab. 2017 March; 120(3):190-197.
Kasiewicz L N, Biswas S, Beach A, Ren H, Dutta C, Mazzola A M, Rohde E, Chadwick A, Cheng C, Garcia S P, Iyer S, Matsumoto Y, Khera A V, Musunuru K, Kathiresan S, Malyala P, Rajeev K G, Bellinger A M. GalNAc-Lipid nanoparticles enable non-LDLR dependent hepatic delivery of a CRISPR base editing therapy. Nat Commun. 2023 May 15; 14(1):2776.
Kim D, Kim D E, Lee G, Cho S I, Kim J S. Genome-wide target specificity of CRISPR RNA-guided adenine base editors. Nat Biotechnol. 2019 April; 37(4):430-435.
Kingwell K. Base editors hit the clinic. Nat. Rev. Drug Discov. 21, 545-547 (2022).
Komor A C, Kim Y B, Packer M S, Zuris J A, Liu D R. Programmable editing of a target base in genomic DNA without double-stranded DNA cleavage. Nature. 2016 May 19; 533(7603):420-4.
Leuders S, Wolfgart E, Ott T, du Moulin M, van Teeffelen-Heithoff A, Vogelpohl L, Och U, Marquardt T, Weglage J, Feldmann R, Rutsch F. Influence of PAH Genotype on Sapropterin Response in PKU: Results of a Single-Center Cohort Study. JIMD Rep. 2014; 13:101-9.
Levy H L, Sarkissian C N, Scriver C R. Phenylalanine ammonia lyase (PAL): From discovery to enzyme substitution therapy for phenylketonuria. Mol Genet Metab. 2018 August; 124(4):223-229. Levy J M, Yeh W H, Pendse N, Davis J R, Hennessey E, Butcher R, Koblan L W, Comander J, Liu Q, Liu D R. Cytosine and adenine base editing of the brain, liver, retina, heart and skeletal muscle of mice via adeno-associated viruses. Nat Biomed Eng. 2020 January; 4(1):97-110.
Liang P, Xie X, Zhi S, Sun H, Zhang X, Chen Y, Chen Y, Xiong Y, Ma W, Liu D, Huang J, Songyang Z. Genome-wide profiling of adenine base editor specificity by EndoV-seq. Nat Commun. 2019 Jan. 8; 10(1):67.
Musunuru K, Chadwick A C, Mizoguchi T, Garcia S P, DeNizio J E, Reiss C W, Wang K, Iyer S, Dutta C, Clendaniel V, Amaonye M, Beach A, Berth K, Biswas S, Braun M C, Chen H M, Colace T V, Ganey J D, Gangopadhyay S A, Garrity R, Kasiewicz L N, Lavoie J, Madsen J A, Matsumoto Y, Mazzola A M, Nasrullah Y S, Nneji J, Ren H, Sanjeev A, Shay M, Stahley M R, Fan S H Y, Tam Y K, Gaudelli N M, Ciaramella G, Stolz L E, Malyala P, Cheng C J, Rajeev K G, Rohde E, Bellinger A M, Kathiresan S. In vivo CRISPR base editing of PCSK9 durably lowers cholesterol in primates. Nature. 2021 May; 593(7859):429-434.
Petri K, Kim D Y, Sasaki K E, Canver M C, Wang X, Shah H, Lee H, Homg J E, Clement K, Iyer S, Garcia S P, Guo J A, Newby G A, Pinello L, Liu D R, Aryee M J, Musunuru K, Joung J K, Pattanayak V. Global-scale CRISPR gene editor specificity profiling by ONE-seq identifies population-specific, variant off-target effects. bioRxiv. 2021 Apr. 5.
Regier D S, Greene C L. Phenylalanine Hydroxylase Deficiency. 2017 Jan. 5. In: Adam M P, Ardinger H H, Pagon R A, Wallace S E, Bean L J H, Gripp K W, Mirzaa G M, Amemiya A, editors. GeneReviews® [Internet]. Seattle (WA): University of Washington, Seattle; 1993-2022.
Richter M F, Zhao K T, Eton E, Lapinaite A, Newby G A, Thuronyi B W, Wilson C, Koblan L W, Zeng J, Bauer D E, Doudna J A, Liu D R. Phage-assisted evolution of an adenine base editor with improved Cas domain compatibility and activity. Nat Biotechnol. 2020 July; 38(7):883-891.
Romani C, Manti F, Nardecchia F, Valentini F, Fallarino N, Carducci C, De Leo S, MacDonald A, Palermo L, Leuzzi V. Adult cognitive outcomes in phenylketonuria: explaining causes of variability beyond average Phe levels. Orphanet J Rare Dis. 2019 Nov. 28; 14(1):273.
Rossidis A C, Stratigis J D, Chadwick A C, Hartman H A, Ahn N J, Li H, Singh K, Coons B E, Li L, Lv W, Zoltick P W, Alapati D, Zacharias W, Jain R, Morrisey E E, Musunuru K, Peranteau W H. In utero CRISPR-mediated therapeutic editing of metabolic genes. Nat Med. 2018 October; 24(10):1513-1518.
Rothgangl T, Dennis M K, Lin P J C, Oka R, Witzigmann D, Villiger L, Qi W, Hruzova M, Kissling L, Lenggenhager D, Borrelli C, Egli S, Frey N, Bakker N, Walker J A 2nd, Kadina A P, Victorov D V, Pacesa M, Kreutzer S, Kontarakis Z, Moor A, Jinek M, Weissman D, Stoffel M, van Boxtel R, Holden K, Pardi N, Thöny B, Häberle J, Tam Y K, Semple S C, Schwank G. In vivo adenine base editing of PCSK9 in macaques reduces LDL cholesterol levels. Nat Biotechnol. 2021 August; 39(8):949-957.
Strecker J, Jones S, Koopal B, Schmid-Burgk J, Zetsche B, Gao L, Makarova K S, Koonin E V, Zhang F. Engineering of CRISPR-Cas12b for human genome editing. Nat Commun. 2019 Jan. 22; 10(1):212. doi: 10.1038/s41467-018-08224-4.
Thomas L, Olson A, Romani C. The impact of metabolic control on cognition, neurophysiology, and well-being in PKU: A systematic review and meta-analysis of the within-participant literature. Mol Genet Metab. 2023 January; 138(1):106969.
van Spronsen F J, Blau N, Harding C, Burlina A, Longo N, Bosch A M. Phenylketonuria. Nat Rev Dis Primers. 2021 May 20; 7(1):36.
Villiger L, Grisch-Chan H M, Lindsay H, Ringnalda F, Pogliano C B, Allegri G, Fingerhut R, Haberle J, Matos J, Robinson M D, Thöny B, Schwank G. Treatment of a metabolic liver disease by in vivo genome base editing in adult mice. Nat Med. 2018 October; 24(10):1519-1525.
Vockley J, Andersson H C, Antshel K M, Braverman N E, Burton B K, Frazier D M, Mitchell J, Smith W E, Thompson B H, Berry S A; American College of Medical Genetics and Genomics Therapeutics Committee. Phenylalanine hydroxylase deficiency: diagnosis and management guideline. Genet Med. 2014 February; 16(2):188-200.
Walton R T, Christie K A, Whittaker M N, Kleinstiver B P. Unconstrained genome targeting with near-PAMless engineered CRISPR-Cas9 variants. Science. 2020 Apr. 17; 368(6488):290-296.
Wang L, Breton C, Warzecha C C, Bell P, Yan H, He Z, White J, Zhu Y, Li M, Buza E L, Jantz D, Wilson J M. Long-term stable reduction of low-density lipoprotein in nonhuman primates following in vivo genome editing of PCSK9. Mol Ther. 2021 Jun. 2; 29(6):2019-2029.
Zetsche B, Gootenberg J S, Abudayyeh O O, Slaymaker I M, Makarova K S, Essletzbichler P, Volz S E, Joung J, van der Oost J, Regev A, Koonin E V, Zhang F. Cpf1 is a single RNA-guided endonuclease of a class 2 CRISPR-Cas system. Cell. 2015 Oct. 22; 163(3):759-71.
Zheng Z, Liebers M, Zhelyazkova B, Cao Y, Panditi D, Lynch K D, Chen J, Robinson H E, Shim H S, Chmielecki J, Pao W, Engelman J A, Iafrate A J, Le L P. Anchored multiplex PCR for targeted next-generation sequencing. Nat Med. 2014 December; 20(12):1479-84.

### Embodiments

Embodiment 1. A method for editing a phenylalanine hydroxylase (PAH) encoding polynucleotide comprising a c.1222C>T (p.Arg408Trp) mutation, the method comprising contacting the PAH polynucleotide with a base editor in complex with at least one guide polynucleotide, wherein the base editor comprises a polynucleotide programmable DNA binding domain and an adenosine deaminase domain, and wherein one or more of said guide polynucleotides target said base editor to effect an A·T to G·C alteration of the mutation.

Embodiment 2. The method of embodiment 1, wherein the contacting is in a cell, a eukaryotic cell, a mammalian cell, or human cell.

Embodiment 3. The method of embodiment 1 or 2, wherein the cell is in vivo.

Embodiment 4. The method of embodiment 1 or 2, wherein the cell is ex vivo.

Embodiment 5. The method of any one of embodiments 1-4, wherein the patient has a second mutation in a different allele in a second PAH encoding polynucleotide selected from c.842C>T (p.Pro281Leu), c.1066-11G>A, c.782G>A (p.Arg261Gln), c.728G>A (p.Arg243Gln), c.1315+1G>A, and c.473G>A (p.Arg158Gln).

Embodiment 6. The method of any one of embodiments 1-5, wherein the polynucleotide programmable DNA binding domain is a Streptococcus pyogenes Cas9 (SpCas9) or Staphylococcus aureus Cas9 (SaCas9) or a variant thereof.

Embodiment 7. The method of any one of embodiments 1-6, wherein the polynucleotide programmable DNA binding domain comprises a modified SpCas9 having an altered protospacer-adjacent motif (PAM) specificity.

Embodiment 8. The method of any one of embodiments 1-7, wherein the polynucleotide programmable DNA binding domain is a nuclease inactive or nickase variant.

Embodiment 9. The method of any one of embodiments 1-8, wherein the adenosine deaminase domain is capable of deaminating adenosine in deoxyribonucleic acid (DNA).

Embodiment 10. The method of embodiment 9, wherein the adenosine deaminase is a TadA deaminase or a variant thereof.

Embodiment 11. The method of any one of embodiments 1-10, wherein the base editor is in complex with a single guide RNA (sgRNA) comprising a nucleic acid sequence complementary to a nucleic acid sequence comprising the mutation associated with PKU.

Embodiment 12. A cell produced by introducing into the cell, or a progenitor thereof:
a) a base editor, or a polynucleotide encoding said base editor, to said cell, wherein said base editor comprises a polynucleotide programmable DNA binding domain and an adenosine deaminase domain; and
b) one or more guide polynucleotides that target the base editor to effect an A·T to G·C alteration of a c.1222C>T (p.Arg408Trp) mutation associated with PKU.

Embodiment 13. The cell of embodiment 12, wherein the cell is a hepatocyte.

Embodiment 14. The cell of embodiment 12 or 13, wherein the hepatocyte expresses a PAH polypeptide.

Embodiment 15. The cell of any one of embodiments 12-14, wherein the cell is from a subject having PKU.

Embodiment 16. The cell of any one of embodiments 12-15, wherein the polynucleotide programmable DNA binding domain is a Streptococcus pyogenes Cas9 (SpCas9) or variant thereof.

Embodiment 17. The cell of any one of embodiments 12-16, wherein the polynucleotide programmable DNA binding domain comprises a modified SpCas9 having an altered protospacer-adjacent motif (PAM) specificity.

Embodiment 18. The cell of embodiment 17, wherein the modified SpCas9 has specificity for the nucleic acid sequence 5′-NGC-3′.

Embodiment 19. The cell of embodiment 17, wherein the modified SpCas9 has specificity for the nucleic acid sequence 5′-NCA-3′.

Embodiment 20. The cell of embodiment 17, wherein the modified SpCas9 has specificity for the nucleic acid sequence 5′-NAA-3′.

Embodiment 21. The cell of embodiment 17, wherein the modified SpCas9 has specificity for the nucleic acid sequence 5′-NAG-3′.

Embodiment 22. The cell of embodiment 17, wherein the modified SpCas9 has specificity for the nucleic acid sequence 5′-NGT-3′.

Embodiment 23. The cell of embodiment 17, wherein the modified SpCas9 has specificity for the nucleic acid sequence 5′-NGN-3′.

Embodiment 24. The cell of embodiment 17, wherein the modified SpCas9 has no specificity for a nucleic acid sequence.

Embodiment 25. The cell of any one of embodiments 12-24, wherein the polynucleotide programmable DNA binding domain is a nuclease inactive or nickase variant.

Embodiment 26. The cell of any one of embodiments 12-25, wherein the adenosine deaminase domain is capable of deaminating adenosine in deoxyribonucleic acid (DNA).

Embodiment 27. The cell of any one of embodiments 12-26, wherein the base editor is in complex with a single guide RNA (sgRNA) comprising a nucleic acid sequence complementary to a PAH encoding nucleic acid sequence comprising the mutation associated with PKU.

Embodiment 28. An adenosine base editor/guide polynucleotide set which corrects a mutation causing PKU comprising:
(i) a modified SpCas9 or SaCas9;
(ii) an adenosine deaminase or functional fragment thereof; and
iii) a guide polynucleotide that targets the base editor to effect an A·T to G·C alteration of a c.1222C>T (p.Arg408Trp) mutation associated with PKU.

Embodiment 29. The method of any one of embodiments 1-11, the cell of any one of embodiments 12-27, or the base editor/guide polynucleotide set of embodiment 28, wherein said guide polynucleotide has a sequence of SEQ ID NO: 1, SEQ ID NO: 3, SEQ ID NO: 5, SEQ ID NO: 7, SEQ ID NO: 9, or SEQ ID NO: 11, or is a hybrid gRNA having one of these sequences with at least one DNA nucleotide substitution in the spacer.

Embodiment 30. The method of any one of embodiments 1-11, the cell of any one of embodiments 12-27, or the base editor/guide polynucleotide set of embodiment 28, wherein said guide polynucleotide comprises a nucleic acid sequence complementary to a PAH encoding nucleic acid sequence of SEQ ID NO: 13, SEQ ID NO: 14, SEQ ID NO: 15, SEQ ID NO: 16, SEQ ID NO: 17, or SEQ ID NO: 18.

Embodiment 31. The method of any one of embodiments 1-11, the cell of any one of embodiments 12-27, or the base editor/guide polynucleotide set of embodiment 28, wherein said guide polynucleotide has a sequence of SEQ ID NO: 7, or is a hybrid gRNA having one of these sequences with at least one DNA nucleotide substitution in the spacer.

Embodiment 32. The method of any one of embodiments 1-11, the cell of any one of embodiments 12-27, or the base editor/guide polynucleotide set of embodiment 28, wherein said base editor has a sequence of SEQ ID NO: 20.

Embodiment 33. The method of any one of embodiments 1-11, the cell of any one of embodiments 12-27, or the base editor/guide polynucleotide set of embodiment 28, wherein
i) said guide polynucleotide has a sequence of SEQ ID NO: 7, or is a hybrid gRNA having one of these sequences with at least one DNA nucleotide substitution in the spacer, and
ii) said base editor has a sequence of SEQ ID NO: 20.

Embodiment 34. A method of treating PKU in a subject comprising administering to said subject an effective amount the adenosine base editor/guide polynucleotide set of any of embodiments 27 to 33.

Embodiment 35. The method of embodiment 34, wherein the subject is a mammal or a human.

Embodiment 36. The method of embodiment 34 or 35, comprising delivering the base editor, or polynucleotide encoding said base editor, and said one or more guide polynucleotides to a cell of the subject.

Embodiment 37. The method of any one of embodiments 34-36, wherein the cell is a liver cell.

Embodiment 38. The method of any one of embodiments 34-37, wherein said base editor/guide polynucleotide set are encapsulated in a lipid nanoparticle formulation and delivered to the liver of said subject.

Embodiment 39. The method of embodiment 38 wherein said formulation comprises ionizable cationic lipid, 1,2-distearoyl-sn-glycero-3-phosphocholine, cholesterol, and a PEG-lipid.

Embodiment 40. The method of any one of embodiments 34-38, wherein said base editor and guide polynucleotide are delivered to hepatocytes in a single or dual AAV vector system.

Embodiment 41. The method of any one of embodiments 34-37, wherein said base editor and guide polynucleotide are delivered to hepatocytes in virus-like particles.

Embodiment 42. A transgenic mouse comprising a humanized Pah gene comprising a c.1222C>T (p.Arg408Trp) mutation.

Embodiment 43. The transgenic mouse of embodiment 42, wherein mouse further comprises at least one mutation selected from c.842C>T (p.Pro281Leu), c.1066-11G>A, c.782G>A (p.Arg261Gln), c.728G>A (p.Arg243Gln), c.1315+1G>A, and c.473G>A (p.Arg158Gln).

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for editing a phenylalanine hydroxylase (PAH) encoding polynucleotide comprising a c.1222C>T (p.Arg408Trp) mutation, the method comprising contacting the PAH polynucleotide with a base editor in complex with at least one guide polynucleotide, wherein the base editor comprises a polynucleotide programmable DNA binding domain and an adenosine deaminase domain, and wherein one or more of said guide polynucleotides target said base editor to effect an A·T to G·C alteration of the mutation.

2. The method of claim 1, wherein the contacting is in a cell, a eukaryotic cell, a mammalian cell, or human cell.

3. The method of claim 2, wherein the cell is in vivo or ex vivo.

4. The method of claim 1, wherein the patient has a second mutation in a different allele in a second PAH encoding polynucleotide selected from c.842C>T (p.Pro281 Leu), c.1066-11G>A, c.782G>A (p.Arg261Gln), c.728G>A (p.Arg243Gln), c.1315+1G>A, and c.473G>A (p.Arg158Gln).

5. The method of claim 1, wherein the polynucleotide programmable DNA binding domain is a Streptococcus pyogenes Cas9 (SpCas9) or Staphylococcus aureus Cas9 (SaCas9) or a variant thereof.

6. The method of claim 1, wherein the polynucleotide programmable DNA binding domain comprises a modified SpCas9 having an altered protospacer-adjacent motif (PAM) specificity.

7. The method of claim 1, wherein the polynucleotide programmable DNA binding domain is a nuclease inactive or nickase variant.

8. The method of claim 1, wherein the adenosine deaminase domain is capable of deaminating adenosine in deoxyribonucleic acid (DNA).

9. The method of claim 8, wherein the adenosine deaminase is a TadA deaminase or a variant thereof.

10. The method of claim 1, wherein the base editor is in complex with a single guide RNA (sgRNA) comprising a nucleic acid sequence complementary to a nucleic acid sequence comprising the mutation associated with PKU.

11. The method of claim 6, wherein the modified SpCas9 has no specificity for a nucleic acid sequence or specificity for the nucleic acid sequence 5′-NGC-3′, 5′-NCA-3′, 5′-NAA-3′, 5′-NAG-3′, 5′-NGT-3′, or 5′-NGN-3′.

12. The method of claim 1, wherein said guide polynucleotide has a sequence of SEQ ID NO: 7, SEQ ID NO: 1, SEQ ID NO: 3, SEQ ID NO: 5, SEQ ID NO: 9, or SEQ ID NO: 11, or is a hybrid gRNA having one of these sequences with at least one DNA nucleotide substitution in the spacer.

13. The method of claim 1, wherein said guide polynucleotide comprises a nucleic acid sequence complementary to a PAH encoding nucleic acid sequence of SEQ ID NO: 13, SEQ ID NO: 14, SEQ ID NO: 15, SEQ ID NO: 16, SEQ ID NO: 17, or SEQ ID NO: 18.

14. The method of claim 1, wherein said guide polynucleotide has a sequence of SEQ ID NO: 7, or is a hybrid gRNA having one of these sequences with at least one DNA nucleotide substitution in the spacer.

15. The method of claim 1, wherein said base editor has a sequence of SEQ ID NO: 20.

16. The method of claim 1, wherein
i) said guide polynucleotide has a sequence of SEQ ID NO: 7, or is a hybrid gRNA having one of these sequences with at least one DNA nucleotide substitution in the spacer; and
ii) said base editor has a sequence of SEQ ID NO: 20.

17. An adenosine base editor/guide polynucleotide set which corrects a mutation causing PKU comprising:
(i) a modified SpCas9 or SaCas9;
(ii) an adenosine deaminase or functional fragment thereof; and
iii) a guide polynucleotide that targets the base editor to effect an A·T to G·C alteration of a c.1222C>T (p.Arg408Trp) mutation associated with PKU.

18. The base editor/guide polynucleotide set of claim 17, wherein said guide polynucleotide has a sequence of SEQ ID NO: 1, SEQ ID NO: 3, SEQ ID NO: 5, SEQ ID NO: 7, SEQ ID NO: 9, or SEQ ID NO: 11, or is a hybrid gRNA having one of these sequences with at least one DNA nucleotide substitution in the spacer.

19. The base editor/guide polynucleotide set of claim 17, wherein said guide polynucleotide comprises a nucleic acid sequence complementary to a PAH encoding nucleic acid sequence of SEQ ID NO: 13, SEQ ID NO: 14, SEQ ID NO: 15, SEQ ID NO: 16, SEQ ID NO: 17, or SEQ ID NO: 18.

20. The base editor/guide polynucleotide set of claim 17, wherein
i) said guide polynucleotide has a sequence of SEQ ID NO: 7, or is a hybrid gRNA having one of these sequences with at least one DNA nucleotide substitution in the spacer, and
ii) said base editor has a sequence of SEQ ID NO: 20.


SEQUENCE LISTING (23 sequences)

SEQ ID NO: 1 - RNA, 100 nt, synthetic construct
  source 1..100: mol_type = other RNA
aagggccaaggtattgtggcgttttagagctagaaatagcaagttaaaataaggctagtccgttatcaacttgaaaaagtggcaccgagtcggtgctttt

SEQ ID NO: 2 - DNA, 20 bp, Homo sapiens
  source 1..20: mol_type = other DNA
aagggccaaggtattgtggc

SEQ ID NO: 3 - RNA, 100 nt, synthetic construct
  source 1..100: mol_type = other RNA
agggccaaggtattgtggcagttttagagctagaaatagcaagttaaaataaggctagtccgttatcaacttgaaaaagtggcaccgagtcggtgctttt

SEQ ID NO: 4 - DNA, 20 bp, Homo sapiens
  source 1..20: mol_type = other DNA
agggccaaggtattgtggca

SEQ ID NO: 5 - RNA, 100 nt, synthetic construct
  source 1..100: mol_type = other RNA
gggccaaggtattgtggcaggttttagagctagaaatagcaagttaaaataaggctagtccgttatcaacttgaaaaagtggcaccgagtcggtgctttt

SEQ ID NO: 6 - DNA, 20 bp, Homo sapiens
  source 1..20: mol_type = other DNA
gggccaaggtattgtggcag

SEQ ID NO: 7 - RNA, 100 nt, synthetic construct
  source 1..100: mol_type = other RNA
ggccaaggtattgtggcagcgttttagagctagaaatagcaagttaaaataaggctagtccgttatcaacttgaaaaagtggcaccgagtcggtgctttt

SEQ ID NO: 8 - DNA, 20 bp, Homo sapiens
  source 1..20: mol_type = other DNA
ggccaaggtattgtggcagc

SEQ ID NO: 9 - RNA, 100 nt, synthetic construct
  source 1..100: mol_type = other RNA
gccaaggtattgtggcagcagttttagagctagaaatagcaagttaaaataaggctagtccgttatcaacttgaaaaagtggcaccgagtcggtgctttt

SEQ ID NO: 10 - DNA, 20 bp, Homo sapiens
  source 1..20: mol_type = other DNA
gccaaggtattgtggcagca

SEQ ID NO: 11 - RNA, 100 nt, synthetic construct
  source 1..100: mol_type = other RNA
ccaaggtattgtggcagcaagttttagagctagaaatagcaagttaaaataaggctagtccgttatcaacttgaaaaagtggcaccgagtcggtgctttt

SEQ ID NO: 12 - DNA, 20 bp, Homo sapiens
  source 1..20: mol_type = other DNA
ccaaggtattgtggcagcaa

SEQ ID NO: 13 - DNA, 20 bp, Homo sapiens
  source 1..20: mol_type = genomic DNA
gccacaataccttggccctt

SEQ ID NO: 14 - DNA, 20 bp, Homo sapiens
  source 1..20: mol_type = genomic DNA
tgccacaataccttggccct

SEQ ID NO: 15 - DNA, 20 bp, Homo sapiens
  source 1..20: mol_type = genomic DNA
ctgccacaataccttggccc

SEQ ID NO: 16 - DNA, 20 bp, Homo sapiens
  source 1..20: mol_type = genomic DNA
gctgccacaataccttggcc

SEQ ID NO: 17 - DNA, 20 bp, Homo sapiens
  source 1..20: mol_type = genomic DNA
tgctgccacaataccttggc

SEQ ID NO: 18 - DNA, 20 bp, Homo sapiens
  source 1..20: mol_type = genomic DNA
ttgctgccacaataccttgg

SEQ ID NO: 19 - RNA, 100 nt, synthetic construct; identical to SEQ ID NO: 7
  source 1..100: mol_type = other RNA
  modified_base 1..4: mod_base = OTHER; note = phosphorothioate linkage
  modified_base 97..100: mod_base = OTHER; note = phosphorothioate linkage
  misc_feature 1..3: note = 2'-O-methylation
  misc_feature 29..40: note = 2'-O-methylation
  misc_feature 69..100: note = 2'-O-methylation

SEQ ID NO: 20 - protein, 1587 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKV

SEQ ID NO: 21 - RNA, 20 nt, synthetic construct
  source 1..20: mol_type = other RNA
agcgaacggagaagggccgg

SEQ ID NO: 22 - DNA, 200 bp, synthetic construct
  source 1..200: mol_type = other DNA
aaaagccacttggaactcctccaggataacctgtctttaaatggtgtccttcactggggtccttggttttggtttcaggaactttgctgccacaataccttggcccttctcagttcgctacgacccctacactcaaagggttgaggtcctggacaatactcagcagttgaagattttagctgactccattaatagtaagt

SEQ ID NO: 23 - DNA, 36 bp, Homo sapiens
  source 1..36: mol_type = genomic DNA
actgagaagggccgaggtattgtggcagcaaagttc